(19) United States
(12) Patent Application Publication
(10) Pub. No.: US 2026/0250673 A1
(43) Pub. Date: Aug. 27, 2026

(54) METHODS AND COMPOSITIONS FOR TREATING CHRONIC PAIN DISORDERS

(71) Applicants: INSTITUT NATIONAL DE LA SANTÉ ET DE LA RECHERCHE MÉDICALE, Paris (FR); UNIVERSITÉ DE MONTPELLIER, MONTPELLIER (FR)

(72) Inventors: Stephanie VENTEO, MONTPELLIER (FR); Alexandre PATTYN, MONTPELLIER (FR); Alexandre DERRE, MONTPELLIER (FR); Patrick CARROLL, MONTPELLIER (FR)

(21) Appl. No.: 18/995,519

(22) PCT Filed: Jul. 20, 2023
(86) PCT No.: PCT/EP2023/070115

§ 371 Date: Jan. 16, 2025

(30) Foreign Application Priority Data
Jul. 21, 2022 (EP) 22306083.1

(51) Int. Cl. C12N 15/113; A61P 25/04

(52) U.S. Cl. CPC C12N 15/113; A61P 25/04; C12N 2310/11; C12N 2310/12; C12N 2310/14; C12N 2310/531

(57) ABSTRACT

Inventors have used FXYD7 knock-out mouse line and DRG-specific conditional Knock-out mice to demonstrate that the constitutive or DRG-specific knock-out of the FXYD7 gene specifically alleviate mechanical chronic pain induced by peripheral inflammation, but not by peripheral nerve lesions. They have also showed that: i) FXYD2 and FXYD7 are expressed by distinct and complementary somatosensory neuron subtypes suggesting specific functions in the somatosensory system; ii) FXYD7 is largely dispensable for neuronal differentiation and survival; iii) in FXYD7−/− animals, FXYD7 expression was not affected in the DRG. Accordingly, the present invention relates to a method for treating a subject suffering from a chronic pain disorder comprising a step of administrating said subject with a therapeutically effective amount of an inhibitor of FXYD7.

### FIELD OF THE INVENTION

The invention is in the field of neurology. More particularly, the invention provides methods and compositions comprising an inhibitor of FXYD7 gene expression for treating chronic pain disorders such as inflammatory chronic pain. Also provided herein are methods of administering the composition of the invention by intrathecal injection in a subject in need thereof.

### BACKGROUND OF THE INVENTION

Acute pain sensation, albeit unpleasant, is a normal and essential protective warning signal for the body which generally persists for a relatively short period. In some circumstances however, pain can become chronic and aberrantly last over a long period, well after its initial cause has disappeared.

Chronic pain is characterized by spontaneous pain, hyperalgesia and/or allodynia which reflect dysfunctions of the neuronal circuits forming the somatosensory nervous system. Its causes are multiple and include lesions to peripheral or central nerves (neuropathic chronic pain) and tissue inflammation (inflammatory chronic pain) (1,2). The pathophysiological mechanisms leading to pain chronification are complex, are still not fully understood and largely depend on the etiology. Nevertheless, they have been proposed to involve deep modifications of genes expression, proteins localization and interactions, and/or axonal plasticity which impact the electrophysiological properties and connectivity of somatosensory neurons, including of the primary afferent neurons located in the dorsal root ganglia (DRG). From medical and social points of view, chronic pain represents a major debilitating disorder which concerns up to 20% of the world population, has deleterious consequences on the quality of life and work productivity, and engenders huge costs for the healthcare systems (3,4). Despite continuous advances in this field, current treatments to relieve subjects are only moderately effective and/or often have adverse side effects (5,6). Therefore, it is still a major challenge to decipher the molecular basis of pain chronification in order to eventually develop novel therapeutic approaches with better efficiency and less side effects (7).

In that respect, inventors and other have recently characterized the transmembrane protein FXYD2 as an important molecular determinant involved in the persistence of peripheral neuropathic and inflammatory chronic pain in rodents (8,9). This protein belongs to the so-called FXYD family which contains 7 members (Fxyd1-7). With the possible exception of Fxyd6, FXYD proteins can interact with- and modulate the activity of the catalytic subunit of the Na,K-ATPase pump, though with distinct effects depending on the protein isoform and tissue localization (10). In the somatosensory nervous system, FXYD2 is largely excluded from its central component, but is specifically expressed by restricted subtypes of peripheral somatosensory neurons of the DRG, namely the D-Hair Low-Threshold Mechanoceptors (LTMRs), the C-LTMRs and the Non-Peptidergic (NP) nociceptors (9,11). In the latter population, FXYD2 plays a key role in modulating neuronal excitability, especially after peripheral nerve damages, which certainly contributes to the establishment of a chronic pain state. Indeed, constitutive or acute knock-down of FXYD2 in rodent models of peripheral neuropathic or inflammatory pain, efficiently alleviate their pain symptoms (8,9).

The restricted expression profile of FXYD7 in the DRG leaves open the possibility that other FXYD family members also play important roles in distinct somatosensory neuron subtypes. However, except for FXYD2, only rare information about the expression and function of FXYD proteins in the DRG is available.

### SUMMARY OF THE INVENTION

In a first aspect, the invention relates to an inhibitor of FXYD7 gene expression for use in a method for treating chronic pain disorders in a subject in need thereof. In particular, the invention is defined by claims.

### DETAILED DESCRIPTION OF THE INVENTION

Inventors have used FXYD7 knock-out mouse line and DRG-specific conditional Knock-out mice to demonstrate that the constitutive or DRG-specific knock-out of the FXYD7 gene specifically alleviate mechanical chronic pain induced by peripheral inflammation, but not by peripheral nerve lesions. They have also showed that: i) FXYD2 and FXYD7 are expressed by distinct and complementary somatosensory neuron subtypes suggesting specific functions in the somatosensory system; ii) FXYD7 is largely dispensable for neuronal differentiation and survival; iii) in FXYD7−/− animals, FXYD2 expression was not affected in the DRG.

Taken together, these results show that in contrast to FXYD2, FXYD7 appears dispensable for the maintenance of peripheral neuropathic chronic pain.

Accordingly, their data establish FXYD7 as a new molecular actor involved in peripheral inflammatory chronic mechanical pain.

This makes FXYD7 as a potential promising target for the development of therapeutic strategies to manage chronic pain disorders with relatively low risk of major side-effects.

### Therapeutic Methods and Uses:

In a first aspect, the invention relates to a method for treating a subject suffering from a chronic pain disorder comprising a step of administrating said subject with a therapeutically effective amount of an inhibitor of FXYD7.

In a particular embodiment, the invention relates to an inhibitor of FXYD7 gene expression for use in a method for treating chronic pain disorders in a subject in need thereof.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is mean the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “chronic pain” refers to a pain that lasts for over three months. The pain can be there all the time, or it may come and go. It can happen anywhere in the body. Chronic pain can interfere with your daily activities, such as working or having a social life. It can lead to depression, anxiety and trouble sleeping, which can make the pain worse. The chronic pain is characterized by a spontaneous pain, hyperalgesia and/or allodynia which reflect dysfunctions of the neuronal circuits forming the somatosensory nervous system. Its causes are multiple and include lesions to peripheral or central nerves (neuropathic chronic pain) and tissue inflammation (inflammatory chronic pain). The pathophysiological mechanisms leading to pain chronification are complex, are still not fully understood and largely depend on the etiology.

In a particular embodiment, the chronic pain disorder is inflammatory chronic pain.

As used herein, the terms “chronic inflammatory pain” or “peripheral inflammatory chronic pain” refer to a common symptom of a variety of autoimmune and inflammatory diseases and pathologic conditions, and includes nociceptive pain (related to an injury to body tissues caused by an inflammatory disease or condition), neuropathic pain (related to abnormalities in the nerves, spinal cord, or brain as a result of an inflammatory disease or condition), and psychogenic pain (entirely or mostly related to a psychological effects of an inflammatory disease or condition). Nociceptive pain includes somatic pain, which arises from bone, joint, muscle, skin, or connective tissue, and visceral pain, which arises from visceral organs, such as the gastrointestinal tract and the pancreas.

As used herein, the term “subject” denotes a mammal, such as a rodent, a feline, a canine, and a primate. Particularly, the subject according to the invention is a human. More particularly, the subject according to the invention has or is susceptible to have a chronic pain disorder.

In a particular embodiment, the subject according to the invention has or is susceptible to have inflammatory chronic pain.

As used herein, the term “FXYD domain containing ion transport regulator 7” (also known as “FXYD7”) has its general meaning in the art and the term includes naturally occurring FXYD7 variants and modified forms thereof.

The naturally occurring human FXYD7 gene has the following nucleotide sequence as shown in Genbank Accession number NM_022006.2 and the naturally occurring human FXYD7 protein has the following amino acid sequences as shown in Genbank Accession number NP_071289.1

As used herein, the term “inhibitor of FXYD7” refers to a natural or synthetic compound that has a biological effect to inhibit the activity and/or expression of FXYD7 gene. Typically, such inhibitor blocks the translation of the targeted mRNA by binding thereto and thus preventing protein translation or increasing mRNA degradation, thus decreasing the level of the targeted protein (i.e. FXYD7), and thus activity, in a cell. In a particular embodiment, the inhibitor of FXYD7 directly inhibit the expression of FXYD7 gene.

In a particular embodiment the inhibitor of gene expression is a siRNA, a shRNA, an antisense oligonucleotide or a ribozyme.

In a particular embodiment the inhibitor of gene expression is a siRNA targeting FXYD7, a shRNA targeting FXYD7, an antisense oligonucleotide targeting FXYD7 or a ribozyme targeting FXYD7.

In one embodiment, the inhibitor of FXYD7 according to the invention is a siRNA, and more particularly a siRNA targeting FXYD7.

As used herein, the term “Small inhibitory RNAs (siRNA)”, also referred to as short interfering RNAs (siRNAs) can also function as FXYD7 expression inhibitors for use in the present invention. FXYD7 gene expression can be reduced by treating the subject or cell with a small double stranded RNA (dsRNA), or a vector or construct causing the production of a small double stranded RNA, such that FXYD7 expression is specifically inhibited (i.e. RNA interference or RNAi) by degradation of mRNAs in a sequence specific manner. Methods for selecting an appropriate dsRNA or dsRNA-encoding vector are known in the art for genes whose sequence is known (e.g. see Tuschl, T. et al. (1999); Elbashir, S. M. et al. (2001); Hannon, G J. (2002); McManus, MT. et al. (2002); Brummelkamp, T R. et al. (2002); U.S. Pat. Nos. 6,573,099 and 6,506,559; and International Patent Publication Nos. WO 01/36646, WO 99/32619, and WO 01/68836, each of which is incorporated by reference herein in its entirety).

In another embodiment, the inhibitor of FXYD7 according to the invention is a shRNA, and more particularly a shRNA targeting FXYD7.

As used herein, the term “Short hairpin RNAs (shRNA)” can also function as FXYD7 expression inhibitors for use in the present invention. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound.

In a particular embodiment, the inhibitor of FXYD7 according to the invention is a miRNA, and in particularly a miRNA targeting FXYD7.

As used herein, the term “miRNAs (miR)” can also function as FXYD7 expression inhibitors for use in the present invention. miRNA has its general meaning in the art and refers to microRNA molecules that are generally 21 to 22 nucleotides in length, even though lengths of 19 and up to 23 nucleotides have been reported, and suppress translation of targeted mRNAs. miRNAs are each processed from a longer precursor RNA molecule (“precursor miRNA”). Precursor miRNAs are transcribed from non-protein-encoding genes. The precursor miRNAs have two regions of complementarity that enables them to form a stem-loop-or fold-back-like structure, which is cleaved in animals by a ribonuclease III-like nuclease enzyme called Dicer. The processed miRNA is typically a portion of the stem. The processed miRNA (also referred to as “mature miRNA”) becomes part of a large complex to downregulate, e.g. decrease translation, of a particular target gene.

Multiple miRNAs may be employed to knockdown FXYD7. The miRNAs may be complementary to different target transcripts or different binding sites of a target transcript. Polycistronic transcripts may also be utilized to enhance the efficiency of target gene knockdown. In some embodiments, multiple genes encoding the same miRNAs or different miRNAs may be regulated together in a single transcript, or as separate transcripts in a single vector cassette. In one embodiment, the vector is a viral vector, including but not limited to recombinant adeno-associated viral (rAAV) vectors, lentiviral vectors, retroviral vectors and retrotransposon-based vector systems.

In another embodiment, ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of the targeted mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

In a particular embodiment, the inhibitor of FXYD7 is an antisense nucleic acid, and more particularly antisense nucleic acid targeting FXYD7.

In a particular embodiment, the inhibitor of FXYD7 expression of the invention is based on antisense oligonucleotide constructs targeting FXYD7.

In a particular embodiment, the inhibitor of FXYD7 gene expression is an antisense oligonucleotide, and more particularly antisense oligonucleotide targeting FXYD7.

As used herein, the term “nucleotide” is defined as a modified or naturally occurring deoxyribonucleotide or ribonucleotide. Nucleotides typically include purines and pyrimidines, which include thymidine (T), cytidine (C), guanosine (G), adenosine (A) and uridine (U).

As used herein, the term “oligonucleotide” refers to an oligomer of the nucleotides defined above. The term “oligonucleotide” refers to a nucleic acid sequence, 3′-5′ or 5′-3′ oriented, which may be single- or double-stranded. The oligonucleotide used in the context of the invention may in particular be DNA or RNA. The term also includes “oligonucleotide analog” which refers to an oligonucleotide having (i) a modified backbone structure, e.g., a backbone other than the standard phosphodiester linkage found in natural oligo- and polynucleotides, and (ii) optionally, modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide {e.g., single-stranded RNA or single-stranded DNA). Particularly, analogs are those having a substantially uncharged, phosphorus containing backbone. A substantially uncharged, phosphorus containing backbone in an oligonucleotide analog is one in which a majority of the subunit linkages, e.g., between 50-100%, typically at least 60% to 100% or 75% or 80% of its linkages, are uncharged, and contain a single phosphorous atom.

As used herein, the term “Antisense oligonucleotides”, including antisense RNA molecules and antisense DNA molecules, refers to nucleic acid that directly block the translation of FXYD7 mRNA by binding thereto and thus preventing protein translation or by increasing mRNA degradation, thus decreasing the level of FXYD7 proteins, and thus activity, in a cell. For example, antisense oligonucleotides of at least about 15 bases and complementary to unique regions of the mRNA transcript sequence encoding FXYD7 can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732, each of which is incorporated by reference herein in its entirety).

The antisense RNA that is complementary to the sense target sequence is encoded by a DNA sequence for the production of any of the foregoing inhibitors (e.g., antisense, siRNAs, shRNAs or miRNAs). The DNA encoding double stranded RNA of interest is incorporated into a gene cassette, e.g. an expression cassette in which transcription of the DNA is controlled by a promoter.

In a particular embodiment, the inhibitor of FXYD7 gene expression is an isolated, synthetic or recombinant antisense oligonucleotide targeting the FXYD7 mRNA transcript. The oligonucleotide of the invention can be of any suitable type.

In some embodiments, the oligonucleotide is a RNA oligonucleotide. In some embodiments, the oligonucleotide is a DNA oligonucleotide.

An antisense strand can be constructed in a number of different ways, provided that it is capable of interfering with the expression of a target gene. For example, the antisense strand can be constructed by reverse-complementing the coding region (or a portion thereof) of the target gene relative to its normal orientation for transcription to allow the transcription of its complement, (e.g., RNAs encoded by the antisense and sense gene may be complementary). In some embodiments, the oligonucleotide need not have the same intron or exon pattern as the target gene, and noncoding segments of the target gene may be equally effective in achieving antisense suppression of target gene expression as coding segments such as antisense oligonucleotide (ASO). In some embodiments, the oligonucleotide has the same exon pattern as the target gene such as siRNA and antisense oligonucleotide (ASO).

As used herein, the term “target” or “targeting” refers to an oligonucleotide able to specifically bind to a FYXD7 gene or a FXYD7 mRNA encoding a FXYD7 gene product. In particular, it refers to an oligonucleotide able to inhibit said gene or said mRNA by the methods known to the skilled in the art (e.g. antisense, RNA interference).

According to the invention, the antisense oligonucleotide of the present invention targets an mRNA and/or DNA encoding FXYD7 gene product and is capable of reducing the amount of FXYD7 expression and/or activity in cells.

That is to say, the antisense oligonucleotide comprises a sequence that is at least partially complementary, particularly perfectly complementary, to a region of the sequence of said mRNA, said complementarity being sufficient to yield specific binding under intracellular conditions. As immediately apparent to the skilled in the art, by a sequence that is “perfectly complementary to” a second sequence is meant the reverse complement counterpart of the second sequence, either under the form of a DNA molecule or under the form of a RNA molecule. A sequence is “partially complementary to” a second sequence if there are one or more mismatches.

The antisense oligonucleotide of the present invention that targets a cDNA or mRNA encoding FXYD7 gene (e.g. FXYD7 gene) that can be designed by using the sequence of said mRNA as a basis, e.g. using bioinformatic tools.

Particularly, the antisense oligonucleotide according to the invention is capable of reducing the expression and/or activity of FXYD7 in DRG. Methods for determining whether an oligonucleotide is capable of reducing the expression and/or activity of FXYD7 in cells are known to those skilled in the art.

This can be performed for example be done by analyzing FXYD7 RNA expression such as by RT-qPCR, in situ hybridization or FXYD7 protein expression such as by immunohistochemistry, Western blot, and by comparing FXYD7 protein expression or FXYD7 functional activity in the presence and in the absence of the antisense oligonucleotide to be tested.

In other embodiments, the oligonucleotide is targeted to a translation initiation site (AUG codon), sequences in the coding region (e.g. one or more exons), 5′-untranslated region or 3′-untranslated region of an mRNA. The aim is to interfere with functions of the messenger RNA include all vital functions including translocation of the RNA to the site for protein translation, actual translation of protein from the RNA, splicing or maturation of the RNA and possibly even independent catalytic activity which may be engaged in by the RNA. The overall effect of such interference with the RNA function is to cause interference with protein expression.

In some embodiments, the oligonucleotide is further modified, particularly chemically modified, in order to increase the stability and/or therapeutic efficiency in vivo. The one skilled in the art can easily provide some modifications that will improve the efficacy of the oligonucleotide such as stabilizing modifications (C. Frank Bennett and Eric E. Swayze, RNA Targeting Therapeutics: Molecular Mechanisms of Antisense Oligonucleotides as a Therapeutic PlatformAnnu. Rev. Pharmacol. Toxicol. 2010.50:259-293; Juliano R L. The delivery of therapeutic oligonucleotides. Nucleic Acids Res. 2016 Aug. 19; 44(14):6518-48). In particular, the oligonucleotide used in the context of the invention may comprise modified nucleotides. Chemical modifications may occur at three different sites: (i) at phosphate groups, (ii) on the sugar moiety, and/or (iii) on the entire backbone structure of the oligonucleotide. Typically, chemical modifications include backbone modifications, heterocycle modifications, sugar modifications, and conjugation strategies.

For example the oligonucleotide is be selected from the group consisting of oligodeoxyribonucleotides, oligoribonucleotides, small regulatory RNAs (sRNAs), U7- or U1-mediated ASOs or conjugate products thereof such as peptide-conjugated or nanoparticle-complexed ASOs, chemically modified oligonucleotide by backbone modifications such as morpholinos, phosphorodiamidate morpholino oligomers (Phosphorodiamidate morpholinos, PMO), peptide nucleic acid (PNA), phosphorothioate (PS) oligonucleotides, stereochemically pure phosphorothioate (PS) oligonucleotides, phosphoramidates modified oligonucleotides, thiophosphoramidate-modified oligonucleotides, and methylphosphonate modified oligonucleotides; chemically modified oligonucleotide by heterocycle modifications such as bicycle modified oligonucleotides, Bicyclic Nucleic Acid (BNA), tricycle modified oligonucleotides, tricyclo-DNA-antisense oligonucleotides (ASOs), nucleobase modifications such as 5-methyl substitution on pyrimidine nucleobases, 5-substituted pyrimidine analogues, 2-Thio-thymine modified oligonucleotides, and purine modified oligonucleotides; chemically modified oligonucleotide by sugar modifications such as Locked Nucleic Acid (LNA) oligonucleotides, 2′,4′-Methyleneoxy Bridged Nucleic Acid (BNA), ethylene-bridged nucleic acid (ENA), constrained ethyl (cEt) oligonucleotides, 2′-Modified RNA, 2′- and 4′-modified oligonucleotides such as 2′-O-Me RNA (2′-OMe), 2′-O-Methoxyethyl RNA (MOE), 2′-Fluoro RNA (FRNA), and 4′-Thio-Modified DNA and RNA; chemically modified oligonucleotide by conjugation strategies such as N-acetyl galactosamine (GalNAc) oligonucleotide conjugates such as 5′-GalNAc and 3′-GalNAc ASO conjugates, lipid oligonucleotide conjugates (LASO), cell penetrating peptides (CPP) oligonucleotide conjugates, targeted oligonucleotide conjugates, antibody-oligonucleotide conjugates, polymer-oligonucleotide conjugate such as with PEGylation and targeting ligand; and chemical modifications and conjugation strategies described for example in Bennett and Swayze, 2010 (RNA targeting therapeutics: molecular mechanisms of antisense oligonucleotides as a therapeutic platform. Annu Rev Pharmacol Toxicol. 2010; 50:259-93); Wan and Seth, 2016 (The Medicinal Chemistry of Therapeutic Oligonucleotides. J Med Chem. 2016 Nov. 10; 59(21):9645-9667); Juliano, 2016 (The delivery of therapeutic oligonucleotides. Nucleic Acids Res. 2016 Aug. 19; 44(14):6518-48); Lundin et al., 2015 (Oligonucleotide Therapies: The Past and the Present. Hum Gene Ther. 2015 August; 26(8):475-85); and Prakash, 2011 (An overview of sugar-modified oligonucleotides for antisense therapeutics. Chem Biodivers. 2011 September; 8(9):1616-41). Indeed, for use in vivo, the oligonucleotide may be stabilized. A “stabilized” oligonucleotide refers to an oligonucleotide that is relatively resistant to in vivo degradation (e.g. via an exo- or endo-nuclease). Stabilization can be a function of length or secondary structure. In particular, oligonucleotide stabilization can be accomplished via phosphate backbone modifications, phosphodiester modifications, phosphorothioate (PS) backbone modifications, combinations of phosphodiester and phosphorothioate modifications, thiophosphoramidate modifications, 2′ modifications (2′-O-Me, 2′-O-(2-methoxyethyl) (MOE) modifications and 2′-fluoro modifications), methylphosphonate, methylphosphorothioate, phosphorodithioate, p-ethoxy, and combinations thereof.

In a particular embodiment, the antisense oligonucleotide comprises 2′-O-methoxyethyl(2′-MOE).

In a particular embodiment, the antisense oligonucleotide is lipid-conjugated, known as LASO. In some embodiments, the antisense oligonucleotide of the present invention is modified by substitution at the 3′ or the 5′ end by a moiety comprising at least three saturated or unsaturated, particularly saturated, linear or branched, particularly linear, hydrocarbon chains comprising from 2 to 30 carbon atoms, particularly from 5 to 20 carbon atoms, more particularly from 10 to 18 carbon atoms as described in WO2014/195432.

In some embodiments, the antisense oligonucleotide of the present invention is modified by substitution at the 3′ or the 5′ end by a moiety comprising at least one ketal functional group, wherein the ketal carbon of said ketal functional group bears two saturated or unsaturated, particularly saturated, linear or branched, particularly linear, hydrocarbon chains comprising from 1 to 22 carbon atoms, particularly from 6 to 20 carbon atoms, in particular 10 to 19 carbon atoms, and even more particularly from 12 to 18 carbon atoms as described in WO2014/195430.

For example, the oligonucleotide may be employed as phosphorothioate derivatives (replacement of a non-bridging phosphoryl oxygen atom with a sulfur atom), which have increased resistance to nuclease digestion. 2′-methoxyethyl (MOE) modification (such as the modified backbone commercialized by IONIS Pharmaceuticals) is also effective. Additionally or alternatively, the oligonucleotide of the present invention may comprise completely, partially or in combination, modified nucleotides which are derivatives with substitutions at the 2′ position of the sugar, in particular with the following chemical modifications: O-methyl group (2′-O-Me) substitution, 2-methoxyethyl group (2′-O-MOE) substitution, fluoro group (2′-fluoro) substitution, chloro group (2′-Cl) substitution, bromo group (2′-Br) substitution, cyanide group (2′—CN) substitution, trifluoromethyl group (2′—CF3) substitution, OCF3 group (2′-OCF3) substitution, OCN group (2′-OCN) substitution, O-alkyl group (2′-O-alkyl) substitution, S-alkyl group (2′-S-alkyl) substitution, N-alkyl group (2′-N-akyl) substitution, O-alkenyl group (2′-O-alkenyl) substitution, S-alkenyl group (2′-S-alkenyl) substitution, N-alkenyl group (2′-N-alkenyl) substitution, SOCH3 group (2′-SOCH3) substitution, SO2CH3 group (2′-SO2CH₃) substitution, ONO2 group (2′-ONO2) substitution, NO2 group (2′-NO2) substitution, N3 group (2′-N3) substitution and/or NH2 group (2′—NH2) substitution. Additionally or alternatively, the oligonucleotide of the present invention may comprise completely or partially modified nucleotides wherein the ribose moiety is used to produce locked nucleic acid (LNA), in which a covalent bridge is formed between the 2′ oxygen and the 4′ carbon of the ribose, fixing it in the 3′-endo configuration. These molecules are extremely stable in biological medium, able to activate RNase H such as when LNA are located to extremities (Gapmer) and form tight hybrids with complementary RNA and DNA.

In some embodiments, the oligonucleotide used in the context of the invention comprises modified nucleotides selected from the group consisting of LNA, 2′-OMe analogs, 2′-O-Met, 2′-O-(2-methoxyethyl) (MOE) oligomers, 2′-phosphorothioate analogs, 2′-fluoro analogs, 2′-Cl analogs, 2′-Br analogs, 2′—CN analogs, 2′—CF3 analogs, 2′-OCF3 analogs, 2′-OCN analogs, 2′-O-alkyl analogs, 2′-S-alkyl analogs, 2′-N-alkyl analogs, 2′-O-alkenyl analogs, 2′-S-alkenyl analogs, 2′-N-alkenyl analogs, 2′-SOCH3 analogs, 2′-SO2CH3 analogs, 2′-ONO2 analogs, 2′-NO2 analogs, 2′-N3 analogs, 2′—NH2 analogs, tricyclo (tc)-DNAs, U7 short nuclear (sn) RNAs, tricyclo-DNA-oligoantisense molecules and combinations thereof (U.S. Provisional Patent Application Ser. No. 61/212,384 For: Tricyclo-DNA Antisense Oligonucleotides, Compositions and Methods for the Treatment of Disease, filed Apr. 10, 2009, the complete contents of which is hereby incorporated by reference).

In a particular embodiment, the oligonucleotide is a LNA oligonucleotide. As used herein, the term “LNA” (Locked Nucleic Acid) (or “LNA oligonucleotide”) refers to an oligonucleotide containing one or more bicyclic, tricyclic or polycyclic nucleoside analogues also referred to as LNA nucleotides and LNA analogue nucleotides. LNA oligonucleotides, LNA nucleotides and LNA analogue nucleotides are generally described in International Publication No. WO 99/14226 and subsequent applications; International Publication Nos. WO 00/56746, WO 00/56748, WO 00/66604, WO 01/25248, WO 02/28875, WO 02/094250, WO 03/006475; U.S. Pat. Nos. 6,043,060, 6,268,490, 6,770,748, 6,639,051, and U.S. Publication Nos. 2002/0125241, 2003/0105309, 2003/0125241, 2002/0147332, 2004/0244840 and 2005/0203042, all of which are incorporated herein by reference. LNA oligonucleotides and LNA analogue oligonucleotides are commercially available from, for example, Proligo LLC, 6200 Lookout Road, Boulder, CO 80301 USA.

Other forms of oligonucleotides are oligonucleotide sequences coupled to small nuclear RNA molecules such as U1 or U7 in combination with a viral transfer method based on, but not limited to, lentivirus or adeno-associated virus (Denti, M A, et al, 2008; Goyenvalle, A, et al, 2004).

Other forms of oligonucleotides are peptide nucleic acids (PNA). In peptide nucleic acids, the deoxyribose backbone of oligonucleotides is replaced with a backbone more akin to a peptide than a sugar. Each subunit, or monomer, has a naturally occurring or non-naturally occurring base attached to this backbone. One such backbone is constructed of repeating units of N-(2-aminoethyl)glycine linked through amide bonds. Because of the radical deviation from the deoxyribose backbone, these compounds were named peptide nucleic acids (PNAs) (Dueholm et al., New J. Chem., 1997, 21, 19-31). PNA binds both DNA and RNA to form PNA/DNA or PNA/RNA duplexes. The resulting PNA/DNA or PNA/RNA duplexes are bound with greater affinity than corresponding DNA/DNA, DNA/RNA or RNA/RNA duplexes as determined by Tm's. This high thermal stability might be attributed to the lack of charge repulsion due to the neutral backbone in PNA. The neutral backbone of the PNA also results in the Tm's of PNA/DNA(RNA) duplex being practically independent of the salt concentration. Thus the PNA/DNA(RNA) duplex interaction offers a further advantage over DNA/DNA, DNA/RNA or RNA/RNA duplex interactions which are highly dependent on ionic strength. Homopyrimidine PNAs have been shown to bind complementary DNA or RNA in an anti-parallel orientation forming (PNA)2/DNA(RNA) triplexes of high thermal stability (see, e.g., Egholm, et al., Science, 1991, 254, 1497; Egholm, et al., J. Am. Chem. Soc., 1992, 114, 1895; Egholm, et al., J. Am. Chem. Soc., 1992, 114, 9677). In addition to increased affinity, PNA has also been shown to bind to DNA or RNA with increased specificity. When a PNA/DNA duplex mismatch is melted relative to the DNA/DNA duplex there is seen an 8 to 20° C. drop in the Tm. This magnitude of a drop in Tm is not seen with the corresponding DNA/DNA duplex with a mismatch present. The binding of a PNA strand to a DNA or RNA strand can occur in one of two orientations. The orientation is said to be anti-parallel when the DNA or RNA strand in a 5′ to 3′ orientation binds to the complementary PNA strand such that the carboxyl end of the PNA is directed towards the 5′ end of the DNA or RNA and amino end of the PNA is directed towards the 3′ end of the DNA or RNA. In the parallel orientation the carboxyl end and amino end of the PNA are just the reverse with respect to the 5′-3′ direction of the DNA or RNA. A further advantage of PNA compared to oligonucleotides is that their polyamide backbones (having appropriate nucleobases or other side chain groups attached thereto) is not recognized by either nucleases or proteases and are not cleaved. As a result, PNAs are resistant to degradation by enzymes unlike nucleic acids and peptides. WO92/20702 describes a peptide nucleic acid (PNA) compounds which bind complementary DNA and RNA more tightly than the corresponding DNA. PNA have shown strong binding affinity and specificity to complementary DNA (Egholm, M., et al., Chem. Soc., Chem. Commun., 1993, 800; Egholm, M., et. al., Nature, 1993, 365, 566; and Nielsen, P., et. al. Nucl. Acids Res., 1993, 21, 197). Furthermore, PNA's show nuclease resistance and stability in cell-extracts (Demidov, V. V., et al., Biochem. Pharmacol., 1994, 48, 1309-1313). Modifications of PNA include extended backbones (Hyrup, B., et. al. Chem. Soc., Chem. Commun., 1993, 518), extended linkers between the backbone and the nucleobase, reversal of the amida bond (Lagriffoul, P. H., et. al., Biomed. Chem. Lett., 1994, 4, 1081), and the use of a chiral backbone based on alanine (Dueholm, K. L, et. al., BioMed. Chem. Lett., 1994, 4, 1077). Peptide Nucleic Acids are described in U.S. Pat. Nos. 5,539,082 and 5,539,083. Peptide Nucleic Acids are further described in U.S. patent application Ser. No. 08/686,113.

Typically, the oligonucleotides are obtained by conventional methods well known to those skilled in the art. For example, the oligonucleotide of the invention can be synthesized de novo using any of a number of procedures well known in the art. For example, the b-cyanoethyl phosphoramidite method (Beaucage et al., 1981); nucleoside H-phosphonate method (Garegg et al., 1986; Froehler et al., 1986, Garegg et al., 1986, Gaffney et al., 1988). These chemistries can be performed by a variety of automated nucleic acid synthesizers available in the market. These nucleic acids may be referred to as synthetic nucleic acids. Alternatively, oligonucleotide can be produced on a large scale in plasmids (see Sambrook, et al., 1989). Oligonucleotide can be prepared from existing nucleic acid sequences using known techniques, such as those employing restriction enzymes, exonucleases or endonucleases. Oligonucleotide prepared in this manner may be referred to as isolated nucleic acids.

The one skilled in the art can easily provide some approaches and modifications for enhancing the delivery and the efficacy of oligonucleotides such as chemical modification of the oligonucleotides, lipid- and polymer-based nanoparticles or nanocarriers, ligand-oligonucleotide conjugates by linking oligonucleotides to targeting agents such as carbohydrates, peptides, antibodies, aptamers, lipids or small molecules and small molecules that improve oligonucleotide delivery such as described in Juliano R L. The delivery of therapeutic oligonucleotides. Nucleic Acids Res. 2016 Aug. 19; 44(14):6518-48. Lipophilic conjugates and lipid conjugates include fatty acid-oligonucleotide conjugates; sterol-oligonucleotide conjugates and vitamin-oligonucleotide conjugates.

In a particular embodiment, the oligonucleotide is conjugated to a second molecule. Typically said second molecule is selected from the group consisting of aptamers, antibodies or polypeptides. For example, the oligonucleotide of the present invention may be conjugated to a cell penetrating peptide. Cell penetrating peptides are well known in the art and include for example the TAT peptide (Bechara C, Sagan S. Cell-penetrating peptides: 20 years later, where do we stand? FEBS Lett. 2013 Jun. 19; 587(12):1693-702).

In some embodiments, the oligonucleotide is associated with a carrier or vehicle, e.g., liposomes or micelles, although other carriers could be used, as would be appreciated by one skilled in the art. Liposomes are vesicles made of a lipid bilayer having a structure similar to biological membranes. Such carriers are used to facilitate the cellular uptake or targeting of the oligonucleotide, or improve the oligonucleotide's pharmacokinetic or therapeutic properties. For example, the oligonucleotide of the present invention may also be administered encapsulated in liposomes, pharmaceutical compositions wherein the active ingredient is contained either dispersed or variously present in corpuscles consisting of aqueous concentric layers adherent to lipidic layers. The oligonucleotide, depending upon solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such as diacetylphosphate, stearylamine, or phosphatidic acid, or other materials of a hydrophobic nature. The diameters of the liposomes generally range from about 15 nm to about 5 microns. The use of liposomes as drug delivery vehicles offers several advantages. Liposomes increase intracellular stability, increase uptake efficiency and improve biological activity. Liposomes are hollow spherical vesicles composed of lipids arranged in a similar fashion as those lipids, which make up the cell membrane. They have an internal aqueous space for entrapping water soluble compounds and range in size from 0.05 to several microns in diameter. Several studies have shown that liposomes can deliver nucleic acids to cells and that the nucleic acids remain biologically active. For example, a liposome delivery vehicle originally designed as a research tool, such as Lipofectin, can deliver intact nucleic acid molecules to cells. Specific advantages of using liposomes include the following: they are non-toxic and biodegradable in composition; they display long circulation half-lives; and recognition molecules can be readily attached to their surface for targeting to tissues. Finally, cost-effective manufacture of liposome-based pharmaceuticals, either in a liquid suspension or lyophilized product, has demonstrated the viability of this technology as an acceptable drug delivery system.

In some embodiments, the oligonucleotide is complexed with a complexing agent to increase cellular uptake of oligonucleotides. An example of a complexing agent includes cationic lipids. Cationic lipids can be used to deliver oligonucleotides to cells. The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Particularly, straight chain or branched alkyl or alkene groups have six or more carbon atoms. Alicyclic groups include cholesterol and other steroid groups. Cationic lipids can be prepared with a variety of counterions (anions) including, e.g., Cl—, Br—, I—, F—, acetate, trifluoroacetate, sulfate, nitrite, and nitrate. Examples of cationic lipids include: polyethylenimine, polyamidoamine (PAMAM) starburst dendrimers, Lipofectin (a combination of DOTMA and DOPE), Lipofectase, Lipofectamine, DOPE, Cytofectin (Gilead Sciences, Foster City, Calif.), and Eufectins (JBL, San Luis Obispo, Calif.). Cationic liposomes may comprise the following: N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3p-[N-(N′,N′-dimethylaminoethane)carbamoyl]cholesterol (DC-Chol), 2,3,-dioleyloxy-N-[2 (sperminecarboxamido)ethyl]-N,N-dimethyl-1-propanaminium trifluoroacetate (DOSPA), 1,2-dimyristyloxypropyl-3-dimethy-1-hydroxyethyl ammonium bromide; and dimethyldioctadecylammonium bromide (DDAB). The cationic lipid N-(1-(2,3-dioleyloxy) propyl)-N,N,N-trimethylammonium chloride (DOTMA), for example, was found to increase 1000-fold the antisense effect of a phosphorothioate oligonucleotide. (Vlassov et al., 1994, Biochimica et Biophysica Acta 1197:95-108). Oligonucleotides can also be complexed with, e.g., poly(L-lysine) or avidin and lipids may, or may not, be included in this mixture (e.g., steryl-poly(L-lysine). Cationic lipids have been used in the art to deliver oligonucleotides to cells (see, e.g., U.S. Pat. Nos. 5,855,910; 5,851,548; 5,830,430; 5,780,053; 5,767,099; Lewis et al. 1996. Proc. Natl. Acad. Sci. USA 93:3176; Hope et al. 1998. Molecular Membrane Biology 15:1). Other lipid compositions which can be used to facilitate uptake of the instant oligonucleotides can be used in connection with the claimed methods. In addition to those listed supra, other lipid compositions are also known in the art and include, e.g., those taught in U.S. Pat. Nos. 4,235,871; 4,501,728; 4,837,028; 4,737,323.

In a further embodiment, the inhibitor of FXYD7 is inserted or not into a vector.

In a further embodiment, the present invention relates to a vector for delivery the inhibitor of FXYD7 as described above. In a particular embodiment, the vector according to the invention wherein the inhibitor is a nucleic acid which encodes an inhibitory RNA that specifically binds to FXYD7 mRNA and inhibits expression of FXYD7 in a cell.

In a particular embodiment, the vector according to invention, wherein the inhibitor is a siRNA, or an antisense oligonucleotide as described above.

In a further embodiment, the inhibitor of FXYD7 (e.g. antisense nucleic acid) of the invention may be delivered in vivo alone (naked ASO/LASO) or in association with a vector.

In its broadest sense, a “vector” is any vehicle capable of facilitating the transfer of the oligonucleotide of the invention to the cells. Particularly, the vector transports the nucleic acid to cells with reduced degradation relative to the extent of degradation that would result in the absence of the vector. In general, the vectors useful in the invention include, but are not limited to, naked plasmids, non-viral delivery systems (cationic transfection agents, liposomes, lipid nanoparticles, and the like), phagemids, viruses, other vehicles derived from viral or bacterial sources that have been manipulated by the insertion or incorporation of the oligonucleotide sequences. Viral vectors include, but are not limited to nucleic acid sequences from the following viruses: RNA viruses such as a retrovirus (as for example moloney murine leukemia virus and lentiviral derived vectors), harvey murine sarcoma virus, murine mammary tumor virus, and rous sarcoma virus; adenovirus, adeno-associated virus (AAV); SV40-type viruses; polyoma viruses; Epstein-Barr viruses; papilloma viruses; herpes virus; vaccinia virus; polio virus. One can readily employ other vectors not named but known to the art.

Accordingly, an object of the invention relates to a vector comprising an oligonucleotide sequence that encodes a portion or fragment of FXYD7, or variants thereof.

In another embodiment, the vector of the invention comprises any variant of the oligonucleotide sequence that encodes a portion or fragment of FXYD7.

In another embodiment, the vector of the invention comprises any variant of the oligonucleotide sequence that encodes any variant of FXYD7.

In another embodiment, the invention relates to a vector comprising an antisense oligonucleotide sequence that encodes a portion or fragment of FXYD7, or variants thereof.

In another embodiment, the invention relates to a vector comprising a shRNA sequence that encodes a portion or fragment of the FXYD7, or variants thereof.

In another embodiment, the invention relates to a vector comprising a miRNA sequence that encodes a portion or fragment of FXYD7, or variants thereof.

In another embodiment, the invention relates to a vector comprising a siRNA sequence that encodes a portion or fragment of FXYD7, or variants thereof.

In another embodiment, the invention relates to a vector comprising an antisense oligonucleotide sequence which targets a portion or fragment of FXYD7, or variants thereof.

In another embodiment, the invention relates to a vector comprising an inhibitor of FYXD7 and a promoter.

In another embodiment, the invention relates to a vector comprising an inhibitor of FYXD7 and a U6 promoter or a PolII promoter.

In another embodiment, the invention relates to a vector comprising an inhibitor of FYXD7 and variants thereof and a CAG promoter.

In another embodiment, the invention relates to a vector comprising an oligonucleotide sequence that encodes a portion or fragment of FXYD7, or variants thereof and a CAG promoter.

In another embodiment, the invention relates to a vector comprising a miRNA sequence that encodes a portion or fragment of FXYD7, or variants thereof and a CAG promoter or a PolII promoter.

In another embodiment, the invention relates to a vector comprising a shRNA sequence that encodes a portion or fragment of FXYD7, or variants thereof and a U6 promoter.

The variants include, for instance, naturally-occurring variants due to allelic variations between individuals (e.g., polymorphisms), alternative splicing forms, etc. The term variant also includes genes sequences of the invention from other sources or organisms. Variants are preferably substantially homologous to sequences according to the invention, i.e., exhibit a nucleotide sequence identity of typically at least about 75%, preferably at least about 85%, more preferably at least about 90%, more preferably at least about 95% with sequences of the invention. Variants of the genes of the invention also include nucleic acid sequences, which hybridize to a sequence as defined above (or a complementary strand thereof) under stringent hybridization conditions. Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

In a particular embodiment, the vector use according to the invention is a non-viral vector or a viral vector.

In a particular embodiment, the non-viral vector is a plasmid comprising a nucleic acid sequence that encodes FXYD7.

In another particular embodiment, the vector may a viral vector.

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction.

As used herein, the term “transgene” refers to the antisense oligonucleotide of the invention.

The terms “gene transfer” or “gene delivery” refer to methods or systems for reliably inserting foreign DNA into host cells. Such methods can result in transient expression of non-integrated transferred DNA, extrachromosomal replication and expression of transferred replicons (e.g. episomes), or integration of transferred genetic material into the genomic DNA of host cells.

Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478.

In a particular embodiment, the viral vector may be an adenoviral, a retroviral, a lentiviral, a herpesvirus or an adeno-associated virus (AAV) vectors.

In a particular embodiment, adeno-associated viral (AAV) vectors are employed.

In another embodiment, the invention relates to an adeno-associated virus (AAV) vector comprising an oligonucleotide sequence that encodes a portion or fragment FXYD7, or variants thereof.

In another embodiment, the adeno-associated virus (AAV) vector of the invention comprises any variant of the oligonucleotide sequence which encodes a portion or fragment of FXYD7.

In another embodiment, the invention relates to an adeno-associated virus (AAV) vector comprising an antisense sequence that encodes a portion or fragment of FXYD7, or variants thereof.

In a particular embodiment, the invention relates to an adeno-associated virus (AAV) vector comprising a shRNA sequence that encodes a portion or fragment of FXYD7, or variants thereof.

In a particular embodiment, the invention relates to an adeno-associated virus (AAV) vector comprising a miRNA sequence that encodes a portion or fragment of FXYD7, or variants thereof.

The variants include, for instance, naturally-occurring variants due to allelic variations between individuals (e.g., polymorphisms), alternative splicing forms, etc. The term variant also includes genes sequences of the invention from other sources or organisms. Variants are preferably substantially homologous to sequences according to the invention, i.e., exhibit a nucleotide sequence identity of typically at least about 75%, preferably at least about 85%, more preferably at least about 90%, more preferably at least about 95% with sequences of the invention. Variants of the genes of the invention also include nucleic acid sequences, which hybridize to a sequence as defined above (or a complementary strand thereof) under stringent hybridization conditions. Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

In a particular embodiment, the vector use according to the invention is a non-viral vector or a viral vector.

In a particular embodiment, the non-viral vector is a plasmid comprising a nucleic acid sequence that encodes FXYD7.

In another particular embodiment, the vector may a viral vector.

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction.

In a particular embodiment, the viral vector may be an adenoviral, a retroviral, a lentiviral, a herpesvirus or an adeno-associated virus (AAV) vectors.

In a particular embodiment, adeno-associated viral (AAV) vectors are employed.

In another embodiment, the invention relates to an adeno-associated virus (AAV) vector comprising an oligonucleotide sequence that encodes a portion or fragment FXYD7, or variants thereof.

In another embodiment, the adeno-associated virus (AAV) vector of the invention comprises any variant of the oligonucleotide sequence which encodes a portion or fragment of FXYD7.

In another embodiment, the invention relates to an adeno-associated virus (AAV) vector comprising an antisense sequence that encodes a portion or fragment of FXYD7, or variants thereof.

In a particular embodiment, the invention relates to an adeno-associated virus (AAV) vector comprising a shRNA sequence that encodes a portion or fragment of FXYD7, or variants thereof.

In a particular embodiment, the invention relates to an adeno-associated virus (AAV) vector comprising a miRNA sequence that encodes a portion or fragment of FXYD7, or variants thereof.

In a particular embodiment, the invention relates to an adeno-associated virus (AAV) vector comprising an oligonucleotide sequence that encodes a portion or fragment of FXYD7, or variants thereof.

In a particular embodiment, the invention relates to an adeno-associated virus (AAV) vector comprising an antisense sequence that encodes a portion or fragment of FXYD7, or variants thereof.

In a particular embodiment, the invention relates to an adeno-associated virus (AAV) vector comprising a miRNA sequence that encodes a portion or fragment of FXYD7, or variants thereof.

In a particular embodiment, the invention relates to an adeno-associated virus (AAV) vector comprising a shRNA sequence that encodes a portion or fragment of FXYD7, or variants thereof.

In a particular embodiment, the invention relates to an adeno-associated virus (AAV) vector comprising an oligonucleotide sequence that encodes a portion or fragment of FXYD7 or variants thereof and a CAG promoter.

In a particular embodiment, the invention relates to an adeno-associated virus (AAV) vector comprising an antisense sequence which targets a portion or fragment of FXYD7, or variants thereof and a CAG promoter.

In a particular embodiment, the invention relates to an adeno-associated virus (AAV) vector comprising a miRNA sequence that encodes a portion or fragment of FXYD7 or variants thereof and a CAG promoter.

In a particular embodiment, the invention relates to an adeno-associated virus (AAV) vector comprising a shRNA sequence that encodes a portion or fragment of FXYD7 or variants thereof and a CAG promoter.

In one embodiment, the AAV vector is AAV1, AAV2, AAV3, AAV4, AA5, AAV6, AAV7, AAV8, AAV9, AAV10, AAVrh.10 or any other serotypes of AAV that can infect human, rodents, monkeys or other species.

By an “AAV vector” is meant a vector derived from an adeno-associated virus serotype, including without limitation, AAV-1, AAV-2, AAV-3, AAV-4, AAV-5, AAV6, AAV7, AAV8, AAV9, AAV10, AAVrh. 10, etc. AAV vectors can have one or more of the AAV wild-type genes deleted in whole or part, e.g. the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. ITRs do not need to be the wild-type polynucleotide sequences, and may be altered, e.g, by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the DNA of interest (i.e. the nucleic acid sequences of the invention) and a transcriptional termination region.

In certain embodiments the viral vectors utilized in the compositions and methods of the invention are recombinant adeno-associated virus (rAAV). The rAAV may be of any serotype, modification, or derivative, known in the art, or any combination thereof (e.g., a population of rAAV that comprises two or more serotypes, e.g., comprising two or more of rAAV2, rAAV8, and rAAV9) known in the art. In some embodiments, the rAAV are rAAV1, rAAV2, rAAV3, rAAV4, rAAV5, rAAV6, rAAV7, rAAV8, rAAV9, rAAV10, rAAV-11, rAAV-12, rAAV-13, rAAV-14, rAAV-15, rAAV-16, rAAV.rh8, rAAV.rh10, rAAV.rh20, rAAV.rh39, rAAV.Rh74, rAAV.RHM4-1, AAV.hu37, rAAV.Anc80, rAAV.Anc80L65, rAAV.7m8, rAAV.PHP.B, rAAV2.5, rAAV2tYF, rAAV3B, rAAV.LK03, rAAV.HSC1, rAAV.HSC2, rAAV.HSC3, rAAV.HSC4, rAAV.HSC5, rAAV.HSC6, rAAV.HSC7, rAAV.HSC8, rAAV.HSC9, rAAV.HSC10, rAAV.HSC11, rAAV.HSC12, rAAV.HSC13, rAAV.HSC14, rAAV.HSC15, or rAAV.HSC16, or other rAAV, or combinations of two or more thereof.

In some embodiments, the rAAV used in the compositions and methods of the invention comprise a capsid protein from an AAV capsid serotype selected from AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. In some embodiments, the rAAV comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., vp1, vp2 and/or vp3 sequence of an AAV capsid serotype selected from AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, the AAV that is used in the methods described herein is Anc80 or Anc80L65, as described in Zinn et al., 2015:1056-1068, which is incorporated by reference in its entirety. In certain embodiments, the AAV that is used in the methods described herein comprises one of the following amino acid insertions: LGETTRP (SEQ ID NO: 1) or LALGETTRP (SEQ ID NO: 2), as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is AAV.7m8, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is any AAV disclosed in U.S. Pat. No. 9,585,971, such as AAV-PHP.B. In certain embodiments, the AAV that is used in the methods described herein is any AAV disclosed in U.S. Pat. No. 9,840,719 and WO 2015/013313, such as AAV.Rh74 and RHM4-1, each of which is incorporated herein by reference in its entirety. In certain embodiments, the AAV that is used in the methods described herein is any AAV disclosed in WO 2014/172669, such as AAV rh.74, which is incorporated herein by reference in its entirety. In certain embodiments, the AAV that is used in the methods described herein is AAV2/5, as described in Georgiadis et al., 2016, Gene Therapy 23:857-862 and Georgiadis et al., 2018, Gene Therapy 25:450, each of which is incorporated by reference in its entirety. In certain embodiments, the AAV that is used in the methods described herein is any AAV disclosed in WO 2017/070491, such as AAV2tYF, which is incorporated herein by reference in its entirety. In certain embodiments, the AAV that is used in the methods described herein is AAVLK03 or AAV3B, as described in Puzzo et al., 2017, Sci. Transl. Med. 29(9): 418, which is incorporated by reference in its entirety. In certain embodiments, the AAV that is used in the methods described herein is any AAV disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In certain embodiments, the AAV that is used in the methods described herein is an AAV disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. In some embodiments, the rAAV have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the vp1, vp2 and/or vp3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, the rAAV have a capsid protein disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689, (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10), the contents of each of which is herein incorporated by reference in its entirety. In some embodiments, the rAAV have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the vp1, vp2 and/or vp3 sequence of an AAV capsid disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689 (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; International Patent Application 2017/0051257; Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

In additional embodiments, the rAAV comprise a pseudotyped rAAV. In some embodiments, the pseudotyped rAAV are rAAV2/8 or rAAV2/9 pseudotyped rAAV. Methods for producing and using pseudotyped rAAV are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, the rAAV comprise a capsid containing a capsid protein chimeric of two or more AAV capsid serotypes. In some embodiments, the capsid protein is a chimeric of 2 or more AAV capsid proteins from AAV serotypes selected from AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, a single-stranded AAV (ssAAV) can be used. In certain embodiments, a self-complementary vector, e.g., scAAV, can be used (see, e.g., Wu, 2007, Human Gene Therapy, 18 (2): 171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

In certain embodiments, the recombinant AAV vector used for delivering the transgene have a tropism for cells in the DRG. Such vectors can include non-replicating “rAAV”, particularly those bearing an AAV8 or AAVrh10 capsid are preferred. In certain embodiments, the viral vectors provided herein are AAV9 or AAVrh10 based viral vectors. In certain embodiments, the AAV8 or AAVrh10 based viral vectors provided herein retain tropism for DRG. AAV variant capsids can be used, including but not limited to those described by Wilson in U.S. Pat. No. 7,906,111 which is incorporated by reference herein in its entirety, with AAV/hu.31 and AAV/hu.32 being particularly preferred; as well as AAV variant capsids described by Chatterjee in U.S. Pat. Nos. 8,628,966, 8,927,514 and Smith et al., 2014, Mol Ther 22:1625-1634, each of which is incorporated by reference herein in its entirety.

In some embodiment, the present invention relates to a recombinant adeno-associated virus (rAAV) comprising (i) an expression cassette containing a transgene under the control of regulatory elements and flanked by ITRs, and (ii) an AAV capsid, wherein the transgene encodes an inhibitory RNA that specifically binds FXYD7 mRNA and inhibits expression of FXYD7 in a cell.

Provided in particular embodiments are AAV vectors comprising an artificial genome comprising (i) an expression cassette containing the transgene under the control of regulatory elements and flanked by ITRs; and (ii) a viral capsid that has the amino acid sequence of the AAV capsid protein or is at least 95%, 96%, 97%, 98%, 99% or 99.9% identical to the amino acid sequence of the AAV capsid protein while retaining the biological function of the AAV capsid.

Provided in particular embodiments are AAVrh10 vectors comprising an artificial genome comprising (i) an expression cassette containing the transgene under the control of regulatory elements and flanked by ITRs; and (ii) a viral capsid that has the amino acid sequence of the AAVrh10 capsid protein or is at least 95%, 96%, 97%, 98%, 99% or 99.9% identical to the amino acid sequence of the AAVrh10 capsid protein while retaining the biological function of the AAVrh10capsid. In certain embodiments, the encoded AAVrh10 capsid has the sequence of SEQ ID NO: 81 set forth in U.S. Pat. No. 9,790,427 which is incorporated by reference herein in its entirety, with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAVrh10 capsid.

The control elements are selected to be functional in a mammalian cell. The resulting construct which contains the operatively linked components is flanked by (5′ and 3′) functional AAV ITR sequences. By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a polynucleotide sequence interposed between two flanking ITRs into a mammalian cell genome. The polynucleotide sequences of AAV ITR regions are known. As used herein, an “AAV ITR” does not necessarily comprise the wild-type polynucleotide sequence, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAV6, AAV7, AAV8, AAV9, AAV10, AAVrh.10, etc. Furthermore, 5′ and 3′ ITRs which flank a selected polynucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV-1, AAV-2, AAV-3, AAV-4, AAV 5, AAV6, AAV7, AAV8, AAV9, AAV10, AAVrh. 10, etc. Furthermore, 5′ and 3′ ITRs which flank a selected polynucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

Particular embodiments are vectors derived from AAV serotypes having tropism for and high transduction efficiencies in cells of the mammalian DRG. A review and comparison of transduction efficiencies of different serotypes is provided in this patent application. In certain examples, AAV2, AAV5, AAV8, AAV9 and rh. 10 based vectors direct long-term expression of transgenes in DRG.

In a particular embodiment, adeno-associated viral (AAV) vectors are employed AAV PHP.S vectors (AAV PHP.S) are employed.

As used herein, the term “AAV-PHP.S” also called AAV9-PHP.S is a variant from AAV9 with AAV capsid evolution (AAV capsid peptide display). With enhanced CNS tropism (neurotropic), the AAV-PHP.S variant vector has also been validated in gene delivery in vivo across the blood-brain barrier (BBB) following intravenous infusion. Such vector is known in the art: Chan et al 2017, Nat Neurosci. 2017 August; 20 (8): 1172-1179; doi: 10.1038/nn.4593.

The selected polynucleotide sequence is operably linked to control elements that direct the transcription or expression thereof in the subject in vivo. Such control elements can comprise control sequences normally associated with the selected gene.

Typically the vector of the present invention comprises an expression cassette. The term “expression cassette” refers to a nucleic acid construct comprising nucleic acid elements sufficient for the expression of the nucleic acid molecule of the present invention. Typically the nucleic acid molecule encodes a heterologous gene and may also include suitable regulatory elements. The heterologous gene refers to a transgene that encodes an RNA of interest.

One or more expression cassettes may be employed. Each expression cassette may comprise at least a promoter sequence operably linked to a sequence encoding the RNA of interest. Each expression cassette may consist of additional regulatory elements, spacers, introns, UTRs, polyadenylation site, and the like. In some embodiments, the expression cassette is polycistronic with respect to the transgenes encoding e.g. two or more miRNAs. In other embodiments the expression cassette comprises a promoter, a nucleic acid encoding one or more RNA molecules of interest, and a polyA. In further embodiments, the expression cassette comprises 5′-promoter sequence, a sequence encoding a first RNA of interest, a sequence encoding a second RNA of interest, and a polyadenylation sequence-3′.

In some embodiments, an expression cassette may comprise additional elements, for example, an intron, an enhancer, a polyadenylation site, a woodchuck posttranscriptional response element (WPRE), and/or other elements known to affect expression levels of the encoding sequence. Typically, an expression cassette comprises the nucleic acid molecule of the present invention operatively linked to a promoter sequence.

The term “operatively linked” refers to the association of two or more nucleic acid fragments on a single nucleic acid fragment so that the function of one is affected by the other.

For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation.

As used herein, the term “promoter” sequence refers to a polynucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

In some embodiments, the promoter is a heterologous promoter. The term “heterologous promoter”, as used herein, refers to a promoter that is not found to be operatively linked to a given encoding sequence in nature.

Useful heterologous control sequences generally include those derived from sequences encoding mammalian or viral genes. Examples include, but are not limited to, the phophoglycerate kinase (PKG) promoter, CAG (composite of the (CMV) cytomegalovirus enhancer the chicken beta actin promoter (CBA) and the rabbit beta globin intron), U6 promoter, neuronal promoters (Human synapsin 1 (hSyn) promoter, NeuN promoters, CamKII promoter, promoter of Dopamine-1 receptor and Dopamine-2 receptor), the SV40 early promoter, mouse mammary tumor virus LTR promoter; adenovirus major late promoter (Ad MLP); a herpes simplex virus (HSV) promoter, a CMV promoter such as the CMV immediate early promoter region (CMV-IE), rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, CA).

For purposes of the present invention, both heterologous promoters and other control elements, such as DRG-specific and inducible promoters, enhancers and the like, will be of particular use.

An “enhancer” is a polynucleotide sequence that can stimulate promoter activity and may be an innate element of the promoter or a heterologous element inserted to enhance the level or tissue-specificity of a promoter. In some embodiments, the promoter is derived in its entirety from a native gene. In some embodiments, the promoter is composed of different elements derived from different naturally occurring promoters. In some embodiments, the promoter comprises a synthetic polynucleotide sequence. It will be understood by those skilled in the art that different promoters will direct the expression of a gene in different tissues or cell types, or at different stages of development, or in response to different environmental conditions or to the presence or the absence of a drug or transcriptional cofactor. Ubiquitous, cell-type-specific, tissue-specific, developmental stage-specific, and conditional promoters, for example, drug-responsive promoters (e.g. tetracycline-responsive promoters) are well known to those of skill in the art.

In mammalian systems, three kinds of promoters exist and are candidates for construction of the expression vectors: Pol I promoters control transcription of large ribosomal RNAs; Pol II promoters control the transcription of mRNAs (that are translated into protein) and small nuclear RNAs (snRNAs); and Pol III promoters uniquely transcribe small non-coding RNAs. Each has advantages and constraints to consider when designing the construct for expression of the RNAs in vivo. For example, Pol III promoters are useful for synthesizing small interfering RNAs (shRNAs) from DNA templates in vivo. For greater control over tissue specific expression, Pol II promoters are preferred but can only be used for transcription of miRNAs. When a Pol II promoter is used, however, it may be preferred to omit translation initiation signals so that the RNAs function as antisense, siRNA, shRNA or miRNAs and are not translated into peptides in vivo.

The AAV expression vector which harbors the DNA molecule of interest flanked by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U. S. Pat. Nos. 5,173,414 and 5,139,941; International Publications Nos. WO 92/01070 (published 23 Jan. 1992) and WO 93/03769 (published 4 Mar. 1993). Alternatively, AAV ITRs can be excised from the viral genome or from an AAV vector containing the same and fused 5′ and 3′of a selected nucleic acid construct that is present in another vector using standard ligation techniques. AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226. Additionally, chimeric genes can be produced synthetically to include AAV ITR sequences arranged 5′and 3′of one or more selected nucleic acid sequences. Preferred codons for expression of the chimeric gene sequence in mammalian DRG cells can be used, and in certain embodiments codon optimization of the transgene is performed by well-known methods. The complete chimeric sequence is assembled from overlapping oligonucleotides prepared by standard methods. In order to produce AAV virions, an AAV expression vector is introduced into a suitable host cell using known techniques, such as by transfection. A number of transfection techniques are generally known in the art. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles.

For instance, a particular viral vector comprises, in addition to a nucleic acid sequence of the invention, the backbone of AAV vector plasmid with ITR derived from AAV-2, the promoter, such as the mouse PGK (phosphoglycerate kinase) gene or the cytomegalovirus/β-actin hybrid promoter (CAG) consisting of the enhancer from the CMV immediate gene, the promoter, splice donor and intron from the chicken β-actin gene, the splice acceptor from rabbit β-globin, or any neuronal promoter such as the promoter of Dopamine-1 receptor or Dopamine-2 receptor, or the synapsin promoter, with or without the wild-type or mutant form of woodchuck hepatitis virus post-transcriptional regulatory element (WPRE), and a rabbit beta-globin polyA sequence. The viral vector may comprise in addition, a nucleic acid sequence encoding an antibiotic resistance gene such as the genes of resistance ampicillin (AmpR), kanamycin, hygromycin B, geneticin, blasticidin S or puromycin.

In one embodiment, retroviral vectors are employed.

Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types.

In another embodiment, lentiviral vectors are employed.

In a particular embodiment, the invention relates to a lentivirus vector comprising an oligonucleotide sequence that encoding a portion or fragment of FXYD7, or variants thereof.

In another embodiment, the lentivirus vector of the invention comprises any variant of the oligonucleotide sequence which encodes a portion or fragment of FXYD7.

In another embodiment, the lentivirus vector of the invention comprises any variant of the oligonucleotide sequence which encodes for any variant of FXYD7.

In another embodiment, the invention relates to a lentivirus vector comprising an antisense oligonucleotide sequence that encodes a portion or fragment of FXYD7, or variants thereof.

In another embodiment, the invention relates to a lentivirus vector comprising a shRNA sequence that encodes a portion or fragment of FXYD7, or variants thereof.

In another embodiment, the invention relates to a lentivirus vector comprising a miRNA sequence that encodes a portion or fragment of FXYD7, or variants thereof.

In another embodiment, the invention relates to a lentivirus vector comprising an oligonucleotide sequence that encodes a portion or fragment of FXYD7, or variants thereof.

In another embodiment, the invention relates to a lentivirus vector comprising a shRNA sequence that encodes a portion or fragment of FXYD7, or variants thereof and a U6 promoter.

In another embodiment, the invention relates to a lentivirus vector comprising an oligonucleotide sequence that encodes a portion or fragment of FXYD7, or variants thereof and a CAG promoter.

In another embodiment, the invention relates to a lentivirus vector comprising an antisense sequence that encodes a portion or fragment of FXYD7, or variants thereof and a CAG promoter.

In another embodiment, the invention relates to a lentivirus vector comprising a miRNA sequence that encodes a portion or fragment of FXYD7, or variants thereof and a CAG promoter.

In another embodiment, the invention relates to a lentivirus vector comprising a shRNA sequence that encodes a portion or fragment of FXYD7, or variants thereof and a CAG promoter.

In a particular embodiment, the invention relates to a lentivirus vector comprising an antisense oligonucleotide which targets a portion or fragment of FXYD7 and a CAG promoter.

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g., U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and that is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species. Typically, the nucleic acid molecule or the vector of the present invention include “control sequences”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

As used herein the terms “administering” or “administration” refer to the act of injecting or otherwise physically delivering a substance as it exists outside the body (e.g., an inhibitor of FXYD7 such as an ASO) into the subject, such as by, intravenous, intramuscular, enteral, subcutaneous, parenteral, systemic, local, spinal, nasal, topical or epidermal administration (e.g., by injection or infusion). When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof. In a particular embodiment, the administration is performed by a patches, a paste, an ointment, a suspension, a solution or a cream, a gel or a spray. In a particular embodiment, the administration is performed by a cream. As used herein, the terms “intrathecal administration” or “intrathecal injection” refer to an injection into the spinal canal (intrathecal space surrounding the spinal cord). Various techniques may be used including, without limitation, lateral cerebroventricular injection through a burrhole or cisternal or lumbar puncture or the like.

In one embodiment, intrathecal administration is used to deliver an inhibitor of FXYD7 gene expression into the CSF. As used herein, intrathecal administration (also referred to as intrathecal injection) refers to an injection into the spinal canal (intrathecal space surrounding the spinal cord). Various techniques may be used including, without limitation, lateral cerebroventricular injection through a burrhole or cisternal or lumbar puncture or the like. Exemplary methods are described in Lazorthes et al. Advances in Drug Delivery Systems and Applications in Neurosurgery, 143-192 and Omaya et al, Cancer Drug Delivery, 1:169-179, the contents of which are incorporated herein by reference.

According to the invention, an inhibitor of FXYD7 gene expression is injected at any region surrounding the spinal canal.

In some embodiments, the inhibitor of FXYD7 gene expression is injected into the lumbar area or the cisterna magna or intraventricularly into a cerebral ventricle space.

As used herein, the term “lumbar region” or “lumbar area” refers to the area between the third and fourth lumbar (lower back) vertebrae and, more inclusively, the L2-S1 region of the spine. Typically, intrathecal injection via the lumbar region or lumber area is also referred to as “lumbar intrathecal delivery” or “lumbar intrathecal administration.”

The term “cisterna magna” refers to the space around and below the cerebellum via the opening between the skull and the top of the spine. Typically, intrathecal injection via cisterna magna is also referred to as “cisterna magna delivery.”

The term “cerebral ventricle” refers to the cavities in the brain that are continuous with the central canal of the spinal cord. Typically, injections via the cerebral ventricle cavities are referred to as “intraventricular Cerebral (ICV) delivery”.

In particular embodiment, the inhibitor of FXYD7 is administered intravenously.

### Pharmaceutical Compositions:

The inhibitor of FXYD7 for use according to the invention alone and/or combined with classical treatment as described above may be combined with pharmaceutically acceptable excipients, and optionally sustained-release matrices, such as biodegradable polymers, to form pharmaceutical compositions.

Accordingly, in a second aspect, the invention relates to a pharmaceutical composition comprising an inhibitor of FXYD7.

In a particular embodiment, the invention relates to a pharmaceutical composition comprising an inhibitor of FXYD7 for use in a method for treating chronic pain disorder in a subject in need thereof.

In a particular embodiment, the pharmaceutical composition according to the invention wherein the chronic pain disorder is chronic inflammatory pain.

In a particular embodiment, the pharmaceutical composition according to the invention wherein the inhibitor of FXYD7 is siRNA.

In a particular embodiment, the pharmaceutical composition according to the invention wherein the inhibitor of FXYD7 is antisense oligonucleotides.

The inhibitor of FXYD7 and the combined preparation as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, intratechal, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In a particular embodiment, the invention relates to a pharmaceutical composition comprising an inhibitor of FXYD7 and a pharmaceutically acceptable carrier, wherein said pharmaceutical composition is formulated for a direct administration into the peripheral nervous system (PNS) of a subject.

The pharmaceutical compositions and related methods of the invention are useful for delivering an inhibitor of FXYD7 to the CNS of a subject (e.g., intrathecally, intraventricularly or intracisternally) and for the treatment of chronic pain disorder.

In one embodiment, the pharmaceutical composition comprising an inhibitor of FXYD7 and a pharmaceutically acceptable carrier is formulated for intrathecal administration. In one embodiment, the pharmaceutical composition comprising an inhibitor of FXYD7 and a pharmaceutically acceptable carrier is formulated for intravenous administration.

### Method of Screening

In a further aspect, the invention relates to a method of screening a drug suitable for the treatment of chronic pain disorder comprising i) providing a test compound and ii) determining the ability of said test compound to inhibit the activity and/or expression of FXYD7.

Any biological assay well known in the art could be suitable for determining the ability of the test compound to inhibit the activity and/or expression of FXYD7.

In some embodiments, the assay first comprises determining the ability of the test compound to bind to FYXD7. In some embodiments, a population of cells is then contacted and activated so as to determine the ability of the test compound to inhibit the activity of and/or expression of FXYD7.

In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of FXYD7, as determined using in vitro methods described herein, are likely to exhibit similar modulatory capacity in applications in vivo. Typically, the test compound is selected from the group consisting of peptides, petptidomimetics, small organic molecules, aptamers or nucleic acids.

The invention will be further illustrated by the following figures and examples. However, these examples and figures should not be interpreted in any way as limiting the scope of the present invention.

### FIGURES

FIG. 1. Fxyd7 mutants exhibit normal behavioral phenotypes.

A. Relative numbers of neurons in adult L4 DRG in WT and constitutive Fxyd7^{-/-} mutants showing no difference. B. Comparative analyses of the percentage of TrkA+, TrkB+, TrkC+, Ret+, Th+ and Fxyd2+ neuronal subtypes in L4 DRG of WT and constitutive Fxyd7^{-/-} mutants revealing no difference. C-E. Analyses of sensorimotor behaviors using the Grid test (measuring muscle strength), the rotarod test (measuring the time-latency to fall from the rotarod device, shown in seconds) and the Openfield test (measuring the time spent at the periphery and at center of the Openfield arena), revealing no statistical difference between females, males or pooled groups of Fxyd7^{-/-} mutants and WT animals. F. Gait parameters analyzed during the Catwalk test showing average speeds, measured in WT and constitutive Fxyd7^{-/-} mutant males and females. No difference was observed in the average speed between WT and Fxyd7^{-/-} mutant females and males. n.s: not significant.

FIG. 2. Analyses of mechanical and thermal sensitivity and social behaviors in constitutive Fxyd7−/− mutants.

A. Analyses of the mechanical sensitivity using von Frey filaments of female, male or pooled groups of WT and Fxyd7^{-/-} mutants showing no difference. B,C. Analyses of the sensitivity to heat using the Hot Plate (at 53° C.; B) and the Hargreaves tests (C) of females, males or pooled groups of WT and Fxyd7^{-/-} mutants showing no difference. D,E. Analyses of the sensitivity to cold using the Cold Plate (D) and the Acetone tests (E) of female, male or pooled groups of WT and Fxyd7^{-/-} mutants. While mutant males showed no defect in all tests, mutant females exhibited a slight, albeit significant hypersensitivity to cold specifically during the cold plate test. F. Analyses of stress and anxio-depressive behaviors using the Splash test of female, male or pooled groups of WT and Fxyd7^{-/-} mutants. Mutant females and males showed no defect in all tests. n.s: not significant; *: p<0,05.

FIG. 3. Specific role for Fxyd7 in sensory neurons in mechanical pain chronification induced by peripheral inflammation.

A-C. In situ hybridization on adult L4 DRG sections from naive WT animals (A), WT animals that underwent a SNL surgery 21 day (d21) before (B) or WT animals that were injected with CFA 14 days (d14) before (C). Scale bar, 100 μm. D. Time course analysis of the percentage of Fxyd7+ neurons in L4 DRG from WT animals that underwent a Sham surgery or a SNL surgery 7 days, 14 days or 28 days before. Note the transient increase of the percentage of Fxyd7+ neurons observed at day 21 in SNL-WT mice. E. Time course analysis of the percentage of Fxyd7+ neurons in L4 DRG from WT animals that were injected with Saline solution or with CFA 2 days, 7 days or 14 days before, revealing no difference at any time point. F-G. Analysis of mechanical sensitivity assessed by the von Frey test on contralateral (control side) and ipsilateral (lesioned side) hind paws of WT and Fxyd7^{-/-} females (F) and males (G) before and up to 41 days after SNL. Measures on the contralateral hind paws served as control and were largely similar to the baseline established before the SNL surgery over time. Measures on the ipsilateral hind paws revealed hypersensitivity in all groups that were maintained over the whole period of the experiment, revealing no beneficial impact of the Fxyd7 mutation. Black arrows indicate the time of SNL surgery. H-J. Analysis of mechanical sensitivity assessed by the von Frey test on contralateral (contra; control side) and ipsilateral (ipsi; injected side) hind paws of WT and Fxyd7^{-/-} (KO) females (H) and males (G) or of Fxyd7^{flox/flox} (Ctrl) and Adv^{Cre}; Fxyd7^{flox/flox} (cKO) females (J) before and up to 16 days after CFA injections. Measures on the contralateral hind paws served as control and were largely similar to the baseline established before CFA injections over time. Measures on the ipsilateral hind paws initially revealed mechanical hypersensitivity in all groups until day 7. From day 7, however, measures on ipsilateral paws from Fxyd7^{-/-} (KO) females (H) and males (G) as well as from Adv^{Cre}; Fxyd7^{flox/flox} (cKO) females (J) progressively returned to baseline levels by day 11 to 16, thus demonstrating that constitutive or conditional Fxyd7 mutations significantly alleviate inflammation-induced chronic pain symptoms. Black arrows indicate the time of CFA injections. K. Analysis of inflammation-induced acute pain using the Formalin Test of WT and Fxyd7^{-/-} (KO) mice assessed by measuring the reaction time of the animal (paw licking, shaking, chewing) during 40 minutes. In all groups, animals responded exhibited a 2-phases response: a first phase starting quickly after Formalin application and lasting 5 minutes-followed by a relative quiescent period of 15 minutes—and a second phase of reactions lasting 20 minutes. No difference was observed between WT and mutant animals. Black arrow indicates the time of Formalin injections. n.s: not significant; *: p<0,05; **: p<0,01; ***: p<0,001.

FIG. 4. Loss of Fxyd7 function is not beneficial for neuropathic- or inflammation-induced cold hypersensitivity.

A. Time course analysis of cold sensitivity assessed by the Acetone test on contralateral (contra; control side) and ipsilateral (ipsi; lesioned side) hind paws of WT and Fxyd7^{-/-} (KO) females before and up to 40 days after SNL surgery. Measures on the contralateral hind paws served as control and were largely similar to the baseline established before the surgery during the whole experiment. Measures on the ipsilateral hind paws revealed a rapid but transient hypersensitivity to cold in both WT and mutants which returned to baseline levels after 30 days. No impact of the Fxyd7 mutation was observed compared to WT. Black arrows indicate the time of SNL surgery. B. Time course analysis of cold sensitivity assessed by the Acetone test on contralateral (contra; control side) and ipsilateral (ipsi; injected side) hind paws of WT and Fxyd7^{-/-} (KO) females before and up to 22 days after CFA injection. Measures on the contralateral hind paws served as control. Measures on the ipsilateral hind paws revealed a slight and transient hypersensitivity to cold in both WT and mutants which returned to baseline levels after 8 days. No beneficial impact of the Fxyd7 mutation was observed in this experimental paradigm. Black arrows indicate the time of CFA injections.

### EXAMPLES

### Material & Methods

### Animals:

All experiments were approved by the French Ministry of Research (authorization #31343) and performed according to the guidelines of the International Association for the Study of Pain (IASP). The Fxyd7 knock-out mouse line used in this study was generated by the Mouse Biology Program (MBP) of the University of California, Davis, and obtained from the Mutant Mouse Resource and Research Center (MMRRC). These mice were maintained on a C57B16N background. DRG-specific conditional Knock-out mice were obtained by using Advillin-cre males (21) maintained on a C57B16J background. Constitutive and conditional mutations were were confirmed by histological analyses (data not shown). For molecular, histological and behavioral studies, control littermates were used. All animals were housed with a 12/12 dark/light cycle and ad libitum access to water and food.

### Surgical Procedures and Treatments

Spinal Nerve Ligation surgery. Unilateral Spinal nerve ligation (SNL) surgery was performed on animals which were deeply anesthetized with isoflurane. Briefly, the animal was immobilized on ventral position. Skin incision was made at low back level using the pelvis as a landmark, and muscle layers were incised to bring out the spinal nerve associated with the L4 DRG. The L5 vertebral process was localized and the spinal nerve L4 was tightly ligated with a 6.0 silk thread (Ethicon). Other nerves were carefully preserved by avoiding any nerve stretch or contact with surgical tools. The muscle layer was closed by careful apposition and sutured with vicryl 5.0 threads. The skin has been stapled and Vetedin has been applied to prevent any infection.

Inflammatory chronic pain. For inflammatory chronic pain assessment, 20 μL of Complete Freund Adjuvant (CFA) have been injected into the plantar surface of the left hind paw. During this procedure, animals were lightly anesthetized with isoflurane.

Acute inflammatory pain. Animals received a sub-cutaneous injection of 10 μL of 2.5% formalin into the upper face of their left hind paw (25). Animals were recorded for 1 hour and videos have been analyzed manually and the time spent by animals shaking, licking and biting the injected paw has been measured. Data are presented as the response duration each 5 minutes.

### Simple and Double In Situ Hybridization

Simple and double in situ hybridization were performed as previously described (11). Digoxigenin (DIG) or Fluorescein-labeled antisense RNA probes were synthesized using the DIG- or Fluorescein-labelling kit (Roche), respectively. Fxyd7, Fxyd2, Ret, TrkA, TrkB, TrkC, Th, Trpm8 probes were used in this study. Animals were euthanized by cervical dislocation and L3 to L5 DRG and spinal cord were dissected in PBS and fixed overnight in PFA 4% at 4° C. Then they were washed twice in PBS before being put in sucrose 30% overnight until organs drowned. Organs have been then cut using a cryostat (Microm) to perform transverse sections of 14 μm.

### Cell Counting

Numbers of neurons expressing the various molecular markers of sensory neuron subtypes in control and Fxyd7−/− littermates, and Fxyd7+ neurons in SNL-operated or CFA mice were determined by counting cells with neuronal morphology and clearly identifiable nuclei. At least six sections from lumbar DRG were counted from three independent animals for each genotype and conditions treated at the same time, in the same experiment. The number of neurons expressing a given marker for each section has been calculated and expressed as a percentage over the total number of DRG neurons or for a given neuronal population.

### Behavioral Testing on Mice

Prior testing, mice were acclimatized for 30 min in the experimental room with only a soft source of light. Experiments have always been conducted at the same period of the day. Experimenters were blinded to the genotype of the mice.

Locomotor coordination. Locomotor coordination was evaluated with a rotarod accelerating procedure (10-80 rpm for 5 min) and the latency to fall was measured in three different experiments. It has been also evaluated with the catwalk xt apparatus. In this test, animals have to walk through a corridor with a glass floor associated with a camera connected to a software which is able to follow paw position, direction and weight repartition during the walk. Five consecutive runs with at least 4 complete strides were recorded for further analysis using CatWalk software 7.1 which permit to analyze static parameters and dynamic parameters of their strides.

Thermal sensitivity. Thermal sensitivity was determined blindly using different tests. The Hargreaves test, the hot and cold plate test and finally the acetone test. In the case of the Hargreaves test, a radiant heat source (a high-intensity projector lamp) was focused onto the plantar surface of the paw and paw-withdrawal latency was determined. Each paw was tested 3 times with a 10 min interval between each trial, and a maximal cut-off time of 20 s was used to prevent tissue damage. The intensity of the lamp has been set to provoke a reaction after 8 sec in the control group, which is at 35% of the max intensity. For the hot plate test, the animal has been gently put on a plate heated at 53° C. At the first sign of reaction of the animal, including paw licking, shaking or jump, the animal has been removed from the plate and the reaction time has been saved. The same protocol has been used for the cold plate test but this time with a plate cooled at 0° C. Results are the mean of at least 3 experiment for each animal separated by at least 2 days to avoid conditional learning. For the acetone test, 60 μL of acetone has been applied on the paw and the time spent licking, shaking or biting their paw has been recorded with a cut-off time of 45 sec.

Mechanical sensitivity. Mechanical sensitivity has been assessed blindly with Von-Frey filaments, by placing animals on an elevated wire mesh grid and stimulating a specific paw with von Frey filaments by using the “up and down” paradigm (28,29) with a specific calcul grid.

The schedule of the experiment was as follows: after baseline analysis, surgery has been done on mice and tests have been performed one week after surgery for SNL mice and the following day for CFA group. Mice were tested postoperative every two days for the von frey experiment and one time per week for temperature assessment. Experiments were performed on seven males and seven females for each genotype for SNL mice or twelve males and twelve females for CFA experiment.

Anxio-depressive assays. Anxio-depressive like behavior has been assessed with three different tests: the splash test, the novelty suppressed feeding test and the forced swim test. In the Splash test, animals received a splash of 10% sucrose solution on their low back. The time spent grooming at the site exposed to sucrose was recorded during a maximum of 5 min. In the novelty suppressed feeding test, animals were left without food during 24 h prior to the test. During the test a pellet of their standard food has been placed in the center of an arena. The animal was placed in a corner. The time they spent prior to get contact with the pellet has been recorded as well as the time spent prior eating the pellet. In the forced swim test, animals were placed in a 17.5 cm beaker containing 11.5 cm of water at 23° C. to avoid the animal to take support on the floor of the beaker. The time during which the animals stop moving, except for slight movements to stay in position, have been recorded after 2 min and for a maximum of 4 min. Mobility and anxiety-like behaviors have been evaluated using an openfield arena (50×50 cm). Mice were placed in the center of the arena and their movements were recorded with infrared captors during 10 minutes. The time spent in the center (central 30×30 cm) or in periphery (external 10 cm) was analyzed as well as the total distance traveled by each group.

Muscle force test. Muscle force has been assessed using the grid (30). In this test, animals had to hold a grid of different weight starting with a 40 g grid followed by 30, 20 and 10 g grids for a maximum of 30 sec. The time before the animal let the grid fall was recorded three times for each grid. The strength value has been obtained by the following formula:

(40g×best time)+(30g×best time)+(20g×best time)+(10g×best time). Note that animals were trained3times before recording.

### Statistical Analyses

For cell counting, statistical analyses were performed using Unpaired Student's t test. For behavioral studies, group and time effects were validated by two-way ANOVAs for repeated measurements. When ANOVAs showed a significant effect, Bonferroni post-hoc test was used to determine the significance of the differences. For electrophysiological analyses, data were analyzed using a two-way ANOVA, followed by Bonferroni test. Fiber conduction velocities were analyzed using the Mann-Whitney U test. Fiber type distribution was analyzed using the Chi-squared test. P values <0.05 were considered as statistically significant. All data presented are means+/− SEM.

### Results:

In the central nervous system, Fxyd7 expression has been well described in the brain (15) and transcriptomic analyses suggest that it is also present in spinal cord motor neurons (16), a result that we have confirmed here (data not shown). In the peripheral nervous system, Fxyd7 had been detected in subpopulations of large-diameter proprioceptive neurons of the DRG (12,13), although data from single cell RNA-Sequencing suggested that it might be more widely distributed in this structure (14). We thus investigated the expression profile of Fxyd7 in adult DRG through co-labelling experiments using a battery of well-established sensory neuron subtype markers, including TrkB (LTMRs), TrkC (LTMRs and Proprioceptors), Ret (LTMRs and NP-nociceptors), TrkA (peptidergic (P)-nociceptors), TrpM8 (cold-sensing thermoceptors) and Tyrosin hydroxylase (Th; C-LTMRs) (see 17 for review). We found that Fxyd7 was expressed in a large proportion of TrkC+ neurons (82%) which includes proprioceptors and LTMRs subtypes (data not shown), representing 35,8% of the entire Fxyd7-positive (+) population in the DRG. Fxyd7 was also detected in a group of TrkB+ LTMRs (32%), representing 6,7% of the Fxyd7+ contingent (data not shown). In the same line, Fxyd7 was co-expressed in 24,7% of the Ret+ neurons, specifically in the large-diameter Rapidly Adapting (RA)-LTMRs (17-19) (data not shown) representing 37,6% of the Fxyd7+ population (data not shown). It was however virtually excluded from small-diameter Ret+ neurons which consist in NP-nociceptors and C-LTMRs populations (data not shown). Consistent with this, only rare Fxyd7+ neurons co-expressed the C-LTMR marker Th (1,1%) (17,20) (data not shown). In contrast, Fxyd7 was detected in a high proportion of the TrkA+ P-thermo/nociceptors (53%) (data not shown), including in the small group of TrpM8+ cold-sensing neurons (28%) (data not shown). These two overlapping populations represented, respectively, 40% and 8% of the entire Fxyd7+ contingent (data not shown). Altogether, these data show that Fxyd7 is expressed by several classes of somatosensory neurons of the DRG including LTMRs, proprioceptors and P-thermo/nociceptors (data not shown). This thus confirms and extends previous histological and transcriptomic studies (12-14). The expression profile of Fxyd7 in the DRG appeared strikingly complementary to Fxyd2 which is expressed in most, if not all, Ret+ NP-nociceptors, Th+ C-LTMRs and in TrkB+ D-Hair LTMRs (8,9,11). This was confirmed by double-labelling experiments that showed that only 1% of Fxyd2+ neurons also expressed Fxyd7 (data not shown). Moreover, we determined that the combined expression of Fxyd2 and Fxyd7 encompass the vast majority of the DRG neurons contingent, though a small population-mainly of large-diameter neurons-appeared to express neither of them (data not shown). Together, these data show that Fxyd7 and Fxyd2 are expressed by distinct and complementary somatosensory neuron subtypes suggesting specific functions in the somatosensory system.

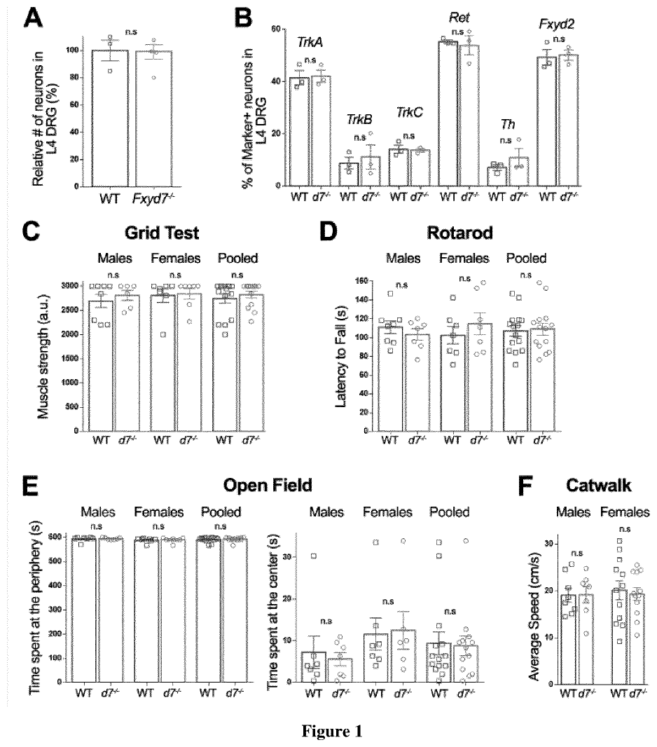

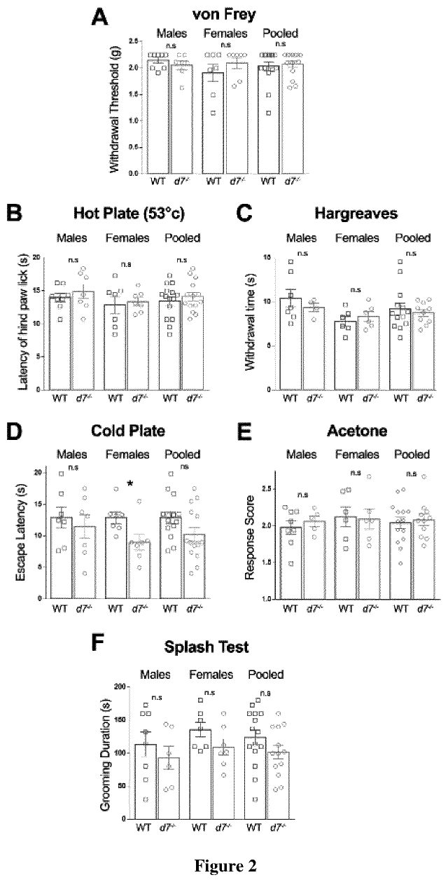

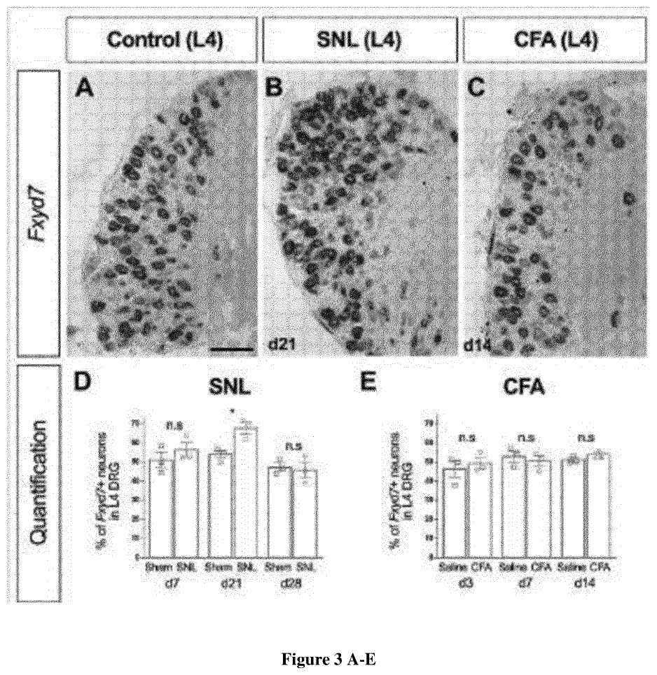

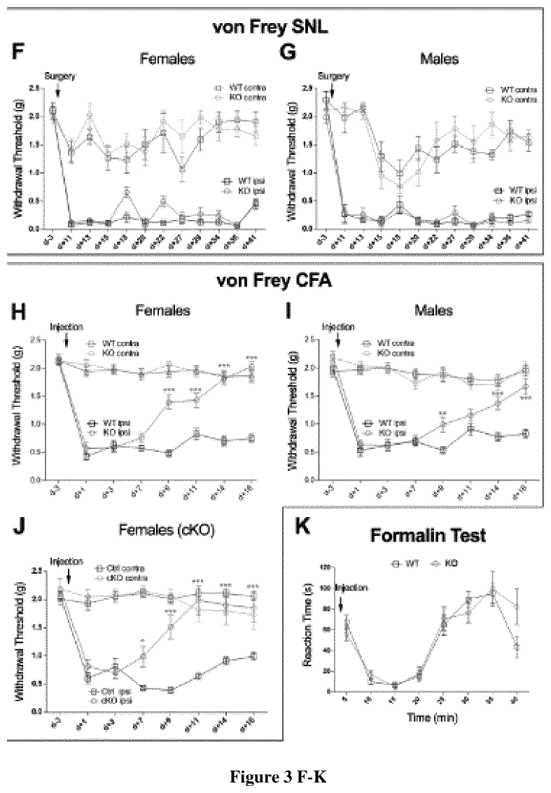

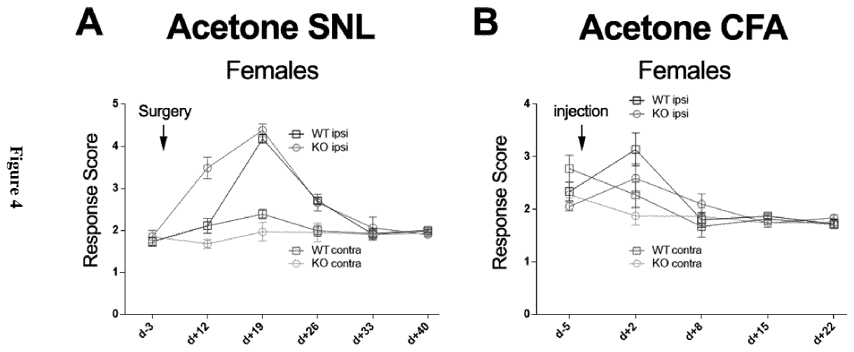

To assess the role of Fxyd7, we took advantage of the existence of a Knock-Out mouse line enabling the generation of either constitutive (Fxyd7−/−) or conditional (Fxyd7flox/flox) mutant alleles (see Materials and Methods data not shown). Constitutive Fxyd7−/− mutants were viable and fertile and histological analyses showed that numbers and molecular identities of somatosensory neurons in their DRG were similar to WT (FIG. 1A, B). This indicates that Fxyd7 is largely dispensable for neuronal differentiation and survival. Moreover, we also determined that in Fxyd7−/− animals, Fxyd2 expression was not affected in the DRG (FIG. 1B).

We next assessed potential behavior defects of Fxyd7−/− mice in physiological conditions, by analyzing cohorts of adult control and mutant males and females. First, since Fxyd7 was expressed in motor and proprioceptive neurons, we evaluated muscle strength, locomotor activity and balance skills of Fxyd7−/− mutants. Using the Grid Test, we did not detect any defect in their muscular strength compared to controls (FIG. 1C). Fxyd7−/− animals also behaved similarly to controls during the rotarod and open field tests (FIG. 1D,E), indicating that their motor activity, coordination and balance abilities were largely unaffected. In line with this, most of the gait parameters recorded during the Catwalk test, including step length, swing or stance phase and velocity, were similar in Fxyd7−/− and control animals (FIG. 1F and data not shown). We nevertheless observed a mild, albeit significant, defect specifically for mutant females. Indeed, we found that the contact surface with the ground of each of their paws was reduced compared to control females (data not shown), which correlated with a higher pressure intensity for each paw (data not shown). This alteration could not be simply explained by an increase of the speed of the mutant females compared to the other cohorts since this parameter was identical for all groups (FIG. 1F). It may rather reflect subtle impairments in their walking abilities possibly due to alterations of their proprioceptive properties, though a motor deficit could not be excluded. To distinguish between a central or a peripheral origin of this defect, we specifically deleted the Fxyd7 gene in somatosensory neurons using the Advillin-Cre (AdvCre) transgenic line (21) (data not shown).

Based on the expression of Fxyd7, we also tested mechanical and thermal sensitivity of Fxyd7−/− animals using a battery of classical somatosensory tests, including the von Frey test (Touch/Pain), the Hot Plate (Heat/Pain) and the Cold Plate and Acetone tests (Cold/Pain). Globally, Fxyd7−/− mutants displayed close to normal behaviors in response to mechanical and thermal stimuli compared to control cohorts (FIG. 2A-E). We nevertheless again observed a relatively mild, albeit significant, phenotype specifically for the mutant females. Indeed, Fxyd7−/− females exhibited hypersensitivity to cold, specifically during the Cold Plate test (FIG. 2D). In contrast, they showed a normal behavior during the Acetone test (FIG. 2E), and they also responded normally to heat as well as to mechanical stimuli (FIG. 2A-C). This suggests a subtle role for Fxyd7 specifically in females for cold sensation, which is consistent with Fxyd7 expression in populations of thermo/nociceptors, including cold-sensing neurons (data not shown). To complement these analyses, we also assessed anxiety- and depression-like behaviors of Fxyd7−/− animals using the Splash test. During these analyses, mutant females and males behaved largely similarly to controls (FIG. 2F). Taken together, these data show that in physiological conditions, Fxyd7−/− mutants only exhibited relatively minor somatosensory deficits, with divergences between males and females. To date, the reasons for the discrepancies between genders remain unclear.

The relatively mild defects of somatosensation observed in Fxyd7−/− mutants echoes the virtually normal sensitivity of Fxyd2−/− mice in physiological condition (8,9). In contrast, when the somatosensory system is challenged in neuropathic or inflammatory contexts, key roles for Fxyd2 in the maintenance of long-lasting pain symptoms have been revealed (8,9). This thus prompted us to analyze putative functions for Fxyd7 in pathophysiological contexts. First, we used the Spinal Nerve Ligation (SNL) model of peripheral neuropathic pain in mice which consisted in the ligature of the nerve at the level of the fourth lumbar DRG (L4). Since Fxyd7 has been previously reported to be up-regulated after sciatic nerve axotomy (12), we first analyzed Fxyd7 expression in the SNL model. In situ hybridization experiments performed on L4 DRG revealed a slight, albeit significant, and transient increase in numbers of Fxyd7-expressing neurons at day (d) 21 which was not maintained at d28 (FIG. 3A, B, D), consistent with previous reports (12). We next analyzed the mechanical sensitivity of control and Fxyd7−/− mutant males and females using von Frey filaments, starting one week after the installation of pain symptoms and over a period of 41 days. In this experimental paradigm, mutant males and females exhibited a pain behavior comparable to control animals, with a rapid installation of mechanical hypersensitivity that was maintained during the whole experiment (FIG. 3F,G). We also assessed their responses to cold using, the Acetone tests. Again, we did not detect any significant difference between mutants and controls over time (data not shown). Taken together, these results show that in contrast to Fxyd2, Fxyd7 appears dispensable for the maintenance of peripheral neuropathic chronic pain.

We next assessed peripheral chronic pain induced by inflammation using the Complete Freund Adjuvant (CFA) model. First, we again performed a comparative time-course analysis of Fxyd7 expression in lumbar DRG of Saline-treated WT and CFA-treated WT animals and showed no significant modification in numbers of Fxyd7+ neurons (FIG. 3C,E). Thus, in contrast to peripheral nerve injury, peripheral inflammation does not appear to alter Fxyd7 expression. We next compared the mechanical sensitivity of groups of CFA-treated WT and constitutive Fxyd7−/− mutant females and males, using the von Frey test. In our hands, in both WT cohorts, CFA injection in the left hind paw rapidly induced mechanical hypersensitivity which lasted for at least 3 weeks (FIG. 3H,I). Also, in both groups of Fxyd7−/− females and males, we initially observed a rapid increase of their mechanical sensitivity similarly to controls (FIG. 3H,I). Strikingly however, mechanical hypersensitivity was not maintained over a long period in the mutants and instead it started to be significantly reverted 9 days after CFA-injections to eventually virtually disappear from day 14 (FIG. 3H,I). It is of note that during the course of this analysis, we again observed few divergences between genders. Indeed, mutant females (FIG. 3H) recovered slightly faster than mutant males (FIG. 3I), though mutant males recovered significantly faster than WT males and females. Again, the reasons for this gender differences remain unclear, but it may reflect the fact that in females, inflammatory pain seems to involve distinct cellular processes than in males (22).

We also analyzed the thermal responses of Fxyd7−/− mutants using the Acetone test. In our hands, CFA-injection in WT cohorts induced a relatively mild and transient hypersensitivity to cold which was eventually reverted after 8 and days (FIG. 4). Mutant females and males behaved similarly to WT during the Acetone test (FIG. 4 and data not shown). Thus, while loss of Fxyd7 function has a beneficial impact on inflammation-induced mechanical pain, it has no effect on thermal pain.

Altogether, these data show that Fxyd7 is specifically critically required for the maintenance of mechanical allodynia induced by inflammation, but not by peripheral nerve lesions. Peripheral neuropathic and inflammatory chronic pain share some common mechanistic features, notably illustrated by the implication of common molecular actors in DRG neurons (23), including Fxyd2 (8,9). However, the specific implication of Fxyd7 in chronic inflammatory pain also highlights the existence of specificity for each type of pain (23), an issue also illustrated by the specific requirement of the receptor tyrosine kinase Flt3 in peripheral neuropathic but not inflammatory-chronic pain (24).

The role of Fxyd7 in the persistence of inflammation-induced mechanical pain prompted us to also assess acute inflammatory pain using the Formalin test. In this model, consistent with previous reports (25), we observed a two phases-response after Formalin injection within approximately a 60 minutes-period: an early transient phase reflecting peripheral nociceptors activation and a second phase arising slightly later, likely reflecting the inflammation process per se (FIG. 3K). Comparative analyses of WT and Fxyd7−/− animals did not reveal any significant difference between groups in any of the 2 phases (FIG. 3K). This indicates that, in contrast to chronic inflammatory pain, Fxyd7 is largely dispensable for acute inflammatory pain.

Impaired maintenance of CFA-induced inflammatory mechanical pain observed in constitutive Fxyd7−/− mutants might reflect a role of Fxyd7 in the somatosensory neurons of the DRG. However, as mentioned above, Fxyd7 is also expressed in the central nervous system (15,16). Therefore, to assess whether Fxyd7 function is required in sensory neurons, we analyzed AdvCre; Fxyd7flox/flox in which Fxyd7 is specifically deleted in the DRG. In this experiments, we compared the mechanical sensitivity of groups of CFA-treated Fxyd7flox/flox (control animals) and CFA-treated AdvCre; Fxyd7flox/flox females, every 2 days using von Frey filaments. We found that CFA-treated AdvCre; Fxyd7flox/flox animals initially exhibited mechanical hypersensitivity soon after injection in a manner comparable to controls (FIG. 3J). However, in contrast to control cohorts, conditional knock-out females did not maintain pain symptoms over time and instead they significantly started to recover after 9 days, similarly to what was observed for constitutive Fxyd7−/− animals (FIG. 3J; see also FIG. 3H). This thus demonstrates a key role for Fxyd7 in somatosensory neurons for the persistence of inflammatory chronic pain. It does not exclude, however, the possibility that it also plays a role in central neurons. Specific deletion of this gene in the central nervous system will help to solve this issue.

Relatively little information is available concerning the role of Fxyd7 in general and in the nervous system in particular. Its ability to bind and influence the functioning of the Na,K-ATPase pump may, at least in part, underlie the effects that we observed at the behavior level (15). Indeed, the activity of this pump is required to maintain and restore the membrane resting potential notably in neurons and, as such, it critically influences neuronal excitability (e.g 26). In models of chronic pain, the electrophysiological properties of somatosensory neurons are deeply modified (27). Fxyd2 has been shown to influence this process in NP-nociceptors after nerve lesions and inflammation (8,9). The complementary expression of Fxyd7 in P-nociceptors and LTMRs open the possibility that Fxyd7 may play similar roles in these populations. This issue will await electrophysiological recordings on Fxyd7−/− mutants in physiological and pathological conditions. Intriguingly, we provide evidence showing that in CFA-induced chronic pain, Fxyd7 appears more specifically required for the maintenance of mechanical pain while it has no beneficial effect on cold hypersensitivity. This may reflect specific and/or divergent functions in distinct neuronal populations depending on the cellular context.

Finally, our data establish Fxyd7 as a new molecular actor involved in peripheral inflammatory chronic mechanical pain. This result, together with the general role of Fxyd2 in neuropathic and inflammatory chronic pain, highlight the importance of the FXYD family in the process of pain chronification. Interestingly, in physiological conditions, loss of Fxyd7 or Fxyd2 function in mice, do not trigger major behavior deficits (8,9, this study). This makes these molecules potential promising targets for the development of therapeutic strategies to manage chronic pain disorders with relatively low risk of major side-effects.

### REFERENCES

Throughout this application, various references describe the state of the art to which this invention pertains. The disclosures of these references are hereby incorporated by reference into the present disclosure.
1) A. I. Basbaum, D. M. Bautista, G. Scherrer, D. Julius, Cellular and molecular mechanisms of pain. Cell 139, 267-284 (2009).
2) R-D. Treede, W. Rief, A. Barke, Q. Aziz, M. Bennett, R. Benoliel, M. Cohen, S. Evers, N. B. Finnerup, M. B. First, M. A. Giamberardino, S. Kaasa, B. Korwisi, E. Kosek, P. Lavand'homme, M. Nicholas, S. Perrot, J. Scholz, S. Schug, B. H. Smith, P. Svensson, J. W. S. Vlaeyen, S-J. Wang, Chronic pain as a symptom or a disease: the IASP Classification of Chronic Pain for the International Classification of Diseases (ICD-11). Pain 160, 19-27 (2019).
3) K. N. Sá, L. Moreira, A. F. Baptista, L. T. Yeng, M. J. Teixeira, R. Galhardoni, D. C. de Andrade, Prevalence of chronic pain in developing countries: systematic review and meta-analysis. Pain Rep. 4, e779 (2019).
4) O. van Hecke, S. K. Austin, R. A. Khan, B. H. Smith, N. Torrance, Neuropathic pain in the general population: a systematic review of epidemiological studies. Pain 155, 654-62 (2013).
5) T. Deer, J. Pope, M. Hanes, G. McDowell. Intrathecal Therapy for Chronic Pain: A Review of Morphine and Ziconotide as Firstline Options. Pain Med. 20, 784-798 (2019).
6) W. Ling, L. Mooney, M. Hillhouse, Prescription opioid abuse, pain and addiction: clinical issues and implications. Drug Alcohol Rev. 30, 300-305 (2011).
7) A. S. Yekkirala, D. P. Roberson, B. P. Bean, C. J. Woolf, Breaking barriers to novel analgesic drug development. Nat. Rev. Drug Discov. 16, 545-564 (2017).
8) S. Ventéo, S. Laffray, C. Wetzel, C. Rivat, F. Scamps, I. Méchaly, L. Bauchet, C. Raoul, E. Bourinet, G. R. Lewin, P. Carroll, A. Pattyn, Fxyd2 regulates Aδ- and C-fiber mechanosensitivity and is required for the maintenance of neuropathic pain. Sci. Rep. 6, 36407 (2016).
9) F. Wang, B. Cai, K. C. Li, X. Y. Hu, Y. J. Lu, Q. Wang, L. Bao, X. Zhang, FXYD2, a γ subunit of Na+,K+-ATPase, maintains persistent mechanical allodynia induced by inflammation. Cell Res. 25, 318-34 (2015).
10) B. Delprat, S. Bibert, K. Geering.«Les protéines FXYD: nouveaux régulateurs de la Na,K-ATPase». Med. Sci. 22, 633 38. (2006)
11) S. Ventéo, S. Bourane, I. Méchaly, C. Sar, O. Abdel Samad, S. Puech, R. Blostein, J. Valmier, A. Pattyn, P. Carroll, Regulation of the Na,K-ATPase gamma-subunit FXYD2 by Runx1 and Ret signaling in normal and injured non-peptidergic nociceptive sensory neurons. PLOS One 7, e29852 (2012).
12) M. Arteaga, R. Gutiérrez, J. Avila, A. Mobasheri, L. diaz-flores, P. Martín-Vasallo.«Regeneration influences expression of the Na+,K+-ATPase subunit isoforms in the rat peripheral nervous system». Neuroscience 129, 691 702. (2004)
13) H. Wu, Haohao, C.Petitpré, P. Fontanet, A. Sharma, C. Bellardita, Rolen M. Quadros, Paulo R. Jannig, et al.«Distinct Subtypes of Proprioceptive Dorsal Root Ganglion Neurons Regulate Adaptive Proprioception in Mice». Nat. Com. 12, 1026. (2019)
14) Usoskin D, et al. Unbiased classification of sensory neuron types by large-scale single-cell RNA sequencing. Nat. Neurosci. 18, 145-153. (2015)
15) P. Béguin, G. Crambert, F. Monnet-Tschudi, M. Uldry, J. Horisberger, H. Garty, K. Geering. FXYD7 is a brain-specific regulator of Na,K-ATPase α1-β isozymes. EMBO J. 21, 3264-3273 (2002).
16) J. Delile, T. Rayon, M. Melchionda, A. Edwards, J. Briscoe, A. Sagner. Single cell transcriptomics reveals spatial and temporal dynamics of gene expression in the developing mouse spinal cord. Development 146, dev173807 (2019).
17) S. Lechner. An update on the spinal and peripheral pathways of pain signaling. Neuroforum 23, A131-A136 (2017).
18 S. Bourane, A. Garces, S. Venteo, A. Pattyn, T. Hubert, A. Fichard, S. Puech, et al. Low-Threshold Mechanoreceptor Subtypes Selectively Express MafA and Are Specified by Ret Signaling. Neuron 64, 857 70 (2009).
19) W. Luo, H. Enomoto, F. Rice, J. Milbrandt, D. Ginty. Molecular Identification of Rapidly Adapting Mechanoreceptors and their Developmental Dependence on Ret Signaling. Neuron 64, 841 56 (2009).
20) M. Delfini, A. Mantilleri, S. Gaillard, J. Hao, A. Reynders, P. Malapert, S. Alonso, A. François, C. Barrere, R. Seal, M. Landry, A. Eschallier, A. Alloui, E. Bourinet, P. Delmas, Y. Le Feuvre, A. Moqrich. TAFA4, a chemokine-like protein, modulates injury-induced mechanical and chemical pain hypersensitivity in mice. Cell Rep. 5, 378-88 (2013).
21) Zurborg S, Piszczek A, Martínez C, Hublitz P, Al Banchaabouchi M, Moreira P, Perlas E, Heppenstall P A. Generation and characterization of an Advillin-Cre driver mouse line. Mol Pain. 7, 66 (2011).
22) A. Dance. Why the Sexes Don't Feel Pain the Same Way. Nature 567, 448 50 (2019).
23) Q. Xu, T. Yaksh. A brief comparison of the pathophysiology of inflammatory versus neuropathic pain. Curr. Opin. in Anaest. 24, 400 407 (2011).
24) C. Rivat, C. Sar, I. Mechaly, J-P. Leyris, L. Diouloufet, C. Sonrier, Y. Philipson, et al. Inhibition of Neuronal FLT3 Receptor Tyrosine Kinase Alleviates Peripheral Neuropathic Pain in Mice. Nat. Comm. 9, 1042 (2018).
25) Rosland, Jan Henrik, Arne Tjølsen, Bjørn Mæhle, et Kjell Hole. The Formalin Test in Mice: Effect of Formalin Concentration. Pain 42, 235 42 (1990).
26) Shao, Li-Rong, Remi Janicot, et Carl E. Stafstrom. Na+-K+-ATPase functions in the developing hippocampus: regional differences in CA1 and CA3 neuronal excitability and role in epileptiform network bursting. Journal of Neurophysiology 125, 1 11 (2021).
27) Finnerup N B, Kuner R, Jensen T S. Neuropathic Pain: From Mechanisms to Treatment. Physiol Rev. 101, 259-301 (2021).
28) Dixon, W. J.«Efficient Analysis of Experimental Observations». Annual Review of Pharmacology and Toxicology 20, 441 62. (1980)
29) Deuis, Jennifer R., Lucie S. Dvorakova, et Irina Vetter.«Methods Used to Evaluate Pain Behaviors in Rodents». Front. in Mol. Neuroscie., 10 (2017).
30) Soulard, Claire, Céline Salsac, Kevin Mouzat, Cécile Hilaire, Julien Roussel, Alexandre Mezghrani, Serge Lumbroso, Cédric Raoul, et Frédérique Scamps.«Spinal Motoneuron TMEM16F Acts at C-Boutons to Modulate Motor Resistance and Contributes to ALS Pathogenesis». Cell Reports 30, 2581-2593 (2020).

1. A method for treating a subject suffering from a chronic pain disorder comprising administrating to said subject a therapeutically effective amount of an inhibitor of FXYD7.

2. The method according to claim 1, wherein the inhibitor of FXYD7 is selected from the group consisting of siRNA, shRNA, an antisense oligonucleotide and a ribozyme.

3. The method according to claim 1, wherein the inhibitor of FXYD7 is inserted or not in a vector.

4. The method according to claim 1, wherein the chronic pain disorder is chronic inflammatory pain.

5. A pharmaceutical composition comprising an inhibitor of FXYD7.

6. A method of treating a chronic pain disorder in a subject in need thereof comprising administering to the patient a therapeutically effective amount of the pharmaceutical composition of claim 5.

7. The method according to claim 6 wherein, the chronic pain disorder is chronic inflammatory pain.

8. The method according to claim 6 wherein the pharmaceutical composition comprises a pharmaceutically acceptable carrier, and wherein said pharmaceutical composition is formulated for a direct administration into the peripheral nervous system of the subject.

9. The method according to claim 6, wherein said pharmaceutical composition is formulated for intrathecal administration.

10. The method according to claim 6, wherein said pharmaceutical composition is formulated for intravenous administration.

11. The pharmaceutical composition according to claim 5, wherein the pharmaceutical composition comprises a pharmaceutically acceptable carrier, and wherein the pharmaceutical composition is formulated for direct administration into the peripheral nervous system of a subject.

12. The pharmaceutical composition according to claim 5, wherein said pharmaceutical composition is formulated for intrathecal administration.

13. The pharmaceutical composition according to claim 5, wherein said pharmaceutical composition is formulated for intravenously administration.


SEQUENCE LISTING (2 sequences)

SEQ ID NO: 1 - protein, 7 aa, synthetic construct
LGETTRP

SEQ ID NO: 2 - protein, 9 aa, synthetic construct
LALGETTRP